SEQUENCE LISTING (12 sequences)

SEQ ID NO: 1 - protein, 12 aa, Artificial Sequence
  Synthesized sequence
GGSVISDNYYWT

SEQ ID NO: 2 - protein, 16 aa, Artificial Sequence
  Synthesized sequence
YIYYRGSTNYNPSLKN

SEQ ID NO: 3 - protein, 12 aa, Artificial Sequence
  Synthesized sequence
DRRTGNSLPFDN

SEQ ID NO: 4 - protein, 11 aa, Artificial Sequence
  Synthesized sequence
SGDKLGDKFAS

SEQ ID NO: 5 - protein, 7 aa, Artificial Sequence
  Synthesized sequence
QDNKRPS

SEQ ID NO: 6 - protein, 9 aa, Artificial Sequence
  Synthesized sequence
QAWDSSIVV

SEQ ID NO: 7 - protein, 122 aa, Artificial Sequence
  Synthesized sequence
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGNSLPFDNWGQGTLVTVSS

SEQ ID NO: 8 - protein, 106 aa, Artificial Sequence
  Synthesized sequence
SYELTQPPSVSVSPGQTAQITCSGDKLGDKFASWYQQKSGQSPVVVIYQDNKRPSGFPERFSGSNSGNTATLTISGTQAMDEADYYCQAWDSSIVVFGGGTKLTVL

SEQ ID NO: 9 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 452 aa, Artificial Sequence
  Synthesized sequence
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGNSLPFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 212 aa, Artificial Sequence
  Synthesized sequence
SYELTQPPSVSVSPGQTAQITCSGDKLGDKFASWYQQKSGQSPVVVIYQDNKRPSGFPERFSGSNSGNTATLTISGTQAMDEADYYCQAWDSSIVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2024/0239885 A1
(43) Pub. Date: Jul. 18, 2024

(54) ANTIBODY CONJUGATE COMPRISING ANTI-P-CADHERIN ANTIBODY AND USES THEREOF

(71) Applicant: WUXI BIOLOGICS IRELAND LIMITED, DUNDALK, CO LOUTH (IE)

(72) Inventors: Yuhong SHEN, Shanghai (CN); Jie LI, Shanghai (CN); Jing LI, Shanghai (CN)

(21) Appl. No.: 18/560,285

(22) PCT Filed: May 12, 2022
(86) PCT No.: PCT/CN2022/092473

§ 371 Date: Nov. 10, 2023

(30) Foreign Application Priority Data
May 13, 2021 (WO) PCT/CN2021/093652

(51) Int. Cl. C07K 16/28; A61K 47/68; A61P 35/00

(52) U.S. Cl. CPC C07K 16/28; A61K 47/68031; A61K 47/68033; A61K 47/6831; A61K 47/6849; A61P 35/00; C07K 2317/73; C07K 2317/77; C07K 2317/92; C07K 2317/94

(57) ABSTRACT

Provided in the present disclosure are anti-P-cadherin antibody-drug conjugates (ADCs) and uses thereof, methods of producing the ADCs as well as methods for validating their functions in vitro and in vivo.

### CROSS REFERENCE

This application claims the benefit of International application PCT/CN2021/093652, filed on May 13, 2021, which is incorporated by reference in its entirety.

### SEQUENCE LISTING

The instant application contains a sequence listing which is hereby incorporated by reference in its entirety.

### FIELD

This application generally relates to antibodies and antibody-drug conjugates. More specifically, the application relates to antibody-drug conjugates against P-cadherin, a method for preparing the same, and the use of the antibody-drug conjugates.

### BACKGROUND

Cadherins family proteins mediate cell-cell adhesions by homophilic interactions between two cadherin molecules at the surface of the respective cells in a cis and/or trans-manner and the cadherin-catenin complex constitutes the main building block of the adherens-type junctions. These complexes also represent a major regulatory mechanism that guides cell fate decisions, influencing cell growth, differentiation, cell motility and survival (Cavallaro and Dejana, Adhesion molecule signaling: not always a sticky business. Nat Rev Mol Cell Biol. 2011 March; 12 (3):189-97).

P-cadherin (Placental-Cadherin, or Cadherin-3, encoded by the CDH3 gene in human) is a 118 kDa glycoprotein classic cadherin. P-cadherin is an 829 amino acid protein with a 26 amino acid long signal sequence and an 803 amino acid propeptide. The mature protein begins at 108 with three distinct domains: five extracellular cadherin repeats (548 aa), which is essential for the creation of lateral dimmers that act together in a zipper-like structure between neighboring cells; single transmembrane region (23 aa); highly conserved cytoplasmic tail (151 aa), an intracellular domain that interacts with catenins, which connect cadherins to the actin cytoskeleton.

P-cadherin is expressed in placenta of mice, also in human placental tissues (lower levels) and several human fetal structures. In adults, it is only expressed in certain tissues, usually co-expressed with E-cadherin, such as the basal layer of epidermis, breast, prostate, mesothelium, ovary, hair follicle, and corneal endothelium (Imai et al., Identification of a novel tumor-associated antigen, cadherin 3 P-cadherin, as a possible target for immunotherapy of pancreatic, gastric, and colorectal cancers. Clin. Cancer Res. 2008, 14, 6487-6495). Major sites of expression indicated by human protein reference database (HPRD: 00227) are endometrium, glomerulus, hair follicle, keratinocytes, mammary myoepithelium, melanocytes, oocytes, spermatozoa, placenta, prostate, retina, serum, and skin.

P-cadherin has been shown to be overexpressed in breast cancer and other tumors and may correlate with poor prognosis. It also showed high expression and positive rate in multiple cancers such as colorectal, NSCLC, gastric cancer and pancreatic cancers. In TCGA database, P-cadherin showed >5 fold higher expression in tumors of: cholangio (10.6×), colon (134× and 104×), esophageal (34×), lung (6.56× and 11.8×), stomach (8.02× and 11.6×) and thyroid (20.3×). P-cadherin may mediate tumor promoting effects including cell invasion, cell motility, stem cell activity and metastases formation in different tissue contexts. P-cadherin gene expression in normal tissues are very low, showing only very weak expression in ovary and mammary gland (GTex data base and literature)

Antibody-based therapy has proved very effective in the treatment of various cancers. In addition to monoclonal antibodies, the use of antibody-drug conjugates for the local delivery of cytotoxic or cytostatic agents allows targeted delivery of the drug moiety to tumors cells rather than normal cells.

There exists a need in the art for anti-P-cadherin antibodies and antibody-drug conjugates. These and other limitations and problems are addressed by the present application.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

The present disclosure provides antibodies against P-cadherin, ADCs comprising the anti-P-cadherin antibodies, and methods for validating the function of ADCs in vitro and in vivo. The ADCs of the present disclosure provide a very potent agent for the treatment of multiple cancers via modulating human immune function.

In some aspects, the present disclosure provides an antibody-drug conjugate (ADC) comprising an antibody or antigen-binding portion thereof conjugated to a drug moiety, wherein the antibody or antigen-binding portion thereof specifically binds to P-cadherin. In some embodiments, the antibody or antigen-binding portion thereof comprises:
(A) one or more heavy chain CDRs (HCDRs) selected from the group consisting of:
(i) a HCDR1 comprising the amino acid sequence of SEQ ID NO: 1;
(ii) a HCDR2 comprising the amino acid sequence of SEQ ID NO: 2; and
(iii) a HCDR3 comprising the amino acid sequence of SEQ ID NO: 3; and
(B) one or more light chain CDRs (LCDRs) selected from the group consisting of:
(i) a LCDR1 comprising the amino acid sequence of SEQ ID NO: 4;
(ii) a LCDR2 comprising the amino acid sequence of SEQ ID NO: 5; and
(iii) a LCDR3 comprising the amino acid sequence of SEQ ID NO: 6.

In some embodiments, the antibody or antigen-binding portion thereof comprises:
(A) a HCDR1 as set forth in SEQ ID NO: 1; a HCDR2 as set forth in SEQ ID NO: 2; and a HCDR3 as set forth in SEQ ID NO: 3; and
(B) a LCDR1 as set forth in SEQ ID NO: 4; a LCDR2 as set forth in SEQ ID NO: 5; and a LCDR3 as set forth in SEQ ID NO: 6.

In some embodiments, the drug moiety as disclosed herein comprises a cytotoxic agent or cytostatic agent selected from a toxin, a chemotherapeutic agent, an antibiotic, a radioactive isotope, and a nucleolytic enzyme. For example, the cytotoxic agent may be selected from maytansinoids such as DM1, DM3, DM4, dolastatins, dolostatin peptidic analogs and derivatives such as auristatins, optionally MMAE and MMAF. In some specific embodiments, the drug moiety comprised in the ADC herein comprises or consists of MMAE.

In some embodiments, the ADC as disclosed herein has the formula Ab-(L-D)p, wherein Ab is the antibody or antigen-binding portion thereof, L is a linker system, D is the drug moiety, and p is a integer from 1 to 20, such as 1, 2, 3, 4, 5, 6, 7, 8, 10, 15 and 20.

In some embodiments, L comprises a linker selected from 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio) pentanoate (SPP), N-succinimidyl 4-(N-maleimidomethyl) cyclohexane-1 carboxylate (SMCC), N-Succinimidyl (4-iodo-acetyl) aminobenzoate (SIAB), and 6-maleimidocaproyl-valine-citrulline-p-aminobenyloxycarbonyl (MC-vc-PAB). For example, the linker is cleavable by a protease. In some specific embodiments, the linker is MC-vc-PAB.

In some specific embodiments, the ADC has the formula Ab-(L-MMAE)p, and p ranges from 1 to 8.

In some embodiments, the linker is attached to the antibody through a thiol group on the antibody.

In some embodiments, the antibody or antigen-binding portion thereof as disclosed herein comprises:
(A) a heavy chain variable region (VH):
(i) comprising the amino acid sequence as set forth in SEQ ID NO: 7;
(ii) comprising an amino acid sequence at least 85%, 90%, or 95% identical to the amino acid sequence as set forth in SEQ ID NO: 7 yet retaining the specific binding affinity to P-cadherin; or
(iii) comprising an amino acid sequence with addition, deletion and/or substitution of one or more (e.g. 1, 2 or 3) amino acids compared with the amino acid sequence as set forth in SEQ ID NO: 7; and/or
(B) a light chain variable region (VL):
(i) comprising the amino acid sequence as set forth in SEQ ID NO: 8;
(ii) comprising an amino acid sequence at least 85%, at least 90%, or at least 95% identical to the amino acid sequence as set forth in SEQ ID NO: 8 yet retaining the specific binding affinity to P-cadherin; or
(iii) comprising an amino acid sequence with addition, deletion and/or substitution of one or more (e.g. 1, 2 or 3) amino acids compared with the amino acid sequence as set forth in SEQ ID NO: 8.

In some embodiments, the addition, deletion and/or substitution of at least one of the amino acids in the VH or VL region is not in any of the CDR sequences, but in the framework (FRW) sequences.

In some embodiments, the isolated antibody or antigen-binding portion thereof as described above further comprises one or more substitutions of the amino acids in the framework sequences, e.g. FRW1, FRW2, FRW3, and/or FRW4 of the VH or VL region.

In some embodiments, the isolated antibody or antigen-binding portion thereof comprises a heavy chain variable region comprising the amino acid sequence as set forth in SEQ ID NO: 7; and a light chain variable region comprising the amino acid sequence as set forth in SEQ ID NO: 8

In some embodiments, the isolated antibody or antigen-binding portion thereof as disclosed herein further comprises a human IgG constant domain, such as a human IgG1, IgG2, IgG3 or IgG4 constant domain, optionally a human IgG1 constant domain or a variant thereof.

In some embodiments, the isolated antibody or antigen-binding portion thereof as disclosed herein is a chimeric antibody, a humanized antibody or a fully human antibody. Preferably, the antibody is a fully human monoclonal antibody.

In some embodiments, the isolated antibody or antigen-binding portion thereof as disclosed herein comprises a heavy chain and a light chain, wherein:
(a) the heavy chain comprises a heavy chain variable region as set forth in SEQ ID NO: 7, and a heavy chain constant region as set forth in SEQ ID NO: 9; and
(b) the light chain comprises a light chain variable region as set forth in SEQ ID NO: 8, 27 or 28, and a light chain constant region as set forth in SEQ ID NO: 10.

In some aspects, the present disclosure is directed to a pharmaceutical composition comprising the ADC as disclosed herein and a pharmaceutically acceptable carrier.

In some aspects, the present disclosure is directed to a method for producing the ADC as defined herein comprising the steps of:
cultivating a host cell comprising a vector encoding the antibody or antigen-binding portion thereof under suitable conditions for the expression of the vector;
isolating the antibody or antigen-binding portion thereof from the host cell; and
conjugating a drug moiety to the antibody or antigen-binding portion thereof.

In some embodiments, the conjugation as described above comprises: reacting a nucleophilic group of a drug moiety with a linker reagent to form drug-linker intermediate D-L, and then reacting D-L with the antibody or antigen-binding portion thereof, alternatively, reacting the antibody with a linker reagent to form antibody-linker intermediate Ab-L, and then reacting Ab-L with an activated drug moiety D, whereby the antibody-drug conjugate is formed. In some embodiments, the DAR of the formed ADCs is in a range of about 1 to about 8, preferably is about 4.

In some embodiments, the drug moiety as disclosed herein comprises a cytotoxic agent or cytostatic agent selected from a toxin, a chemotherapeutic agent, an antibiotic, a radioactive isotope, and a nucleolytic enzyme. For example, the cytotoxic agent may be selected from maytansinoids such as DM1, DM3, DM4, dolastatins, dolostatin peptidic analogs and derivatives such as auristatins, optionally MMAE and MMAF. In some specific embodiments, the drug moiety comprised in the ADC herein comprises or consists of MMAE.

In some aspects, the present disclosure is directed to a method of modulating an P-cadherin-related immune response in a subject, comprising administering the ADC as disclosed herein to the subject such that the P-cadherin-related immune response in the subject is modulated.

In some aspects, the present disclosure is directed to a method for treating or preventing a P-cadherin positive cancer in a subject, comprising administering an effective amount of the ADC or the pharmaceutical composition as disclosed herein to the subject. In some embodiments, said cancer can be selected from breast cancer, lung cancer, colon cancer, ovarian cancer, melanoma, bladder cancer, renal cell carcinoma, liver cancer, prostate cancer, stomach cancer, pancreatic cancer, NSCLC, cervical cancer, esophageal carcinoma, endometrial cancer, skin cancer, head and neck cancer, testis cancer, thyroid cancer, urothelial cancer, non-Hodgkin's lymphoma, chronic lymphocytic leukemia, diffuse large B-cell lymphoma, and multiple myeloma. In some embodiments, said cancer is NSCLC, prostate cancer or colorectal cancer. In some embodiments, said cancer is breast cancer, including breast ductal carcinoma.

In some aspects, the present disclosure is directed to the use of the ADC as disclosed herein in the manufacture of a medicament for diagnosing, treating or preventing P-cadherin positive cancer.

In some aspects, the present disclosure is directed to the ADC as disclosed herein for use in diagnosing, treating or preventing P-cadherin positive cancer.

In some aspects, the present disclosure is directed to kits or devices and associated methods that employ the ADC as disclosed herein, and pharmaceutical compositions as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. This summary is not intended to identify key features or essential features of the claimed subject matter, nor is it intended to be used as an aid in determining the scope of the claimed subject matter.

### BRIEF DESCRIPTION OF THE FIGURES

FIGS. 1A-1B show the HPLC result of W3195-p1-MMAE (A) and BMK4-DM1 (B).

FIGS. 2A-2B show the result of FACS binding assay of ADCs on human P-cadherin expressing HCT-116 cells (A) and NCI-H1650 cells (B).

FIG. 3 shows the serum stability result of W3195-p1-MMAE by FACS binding.

FIGS. 4A-4F show cytotoxicity effect of ADCs on HCC-1954 cells (A), HCC-70 cells (B), HT-29 cells (C), A549 cells (D), MDA-MB-453 cells (E) and NCI-H1650 cells (F).

FIGS. 5A-5B show the internalization ability of ADCs on HCC-1954 cells (A) or NCI-H1650 cells (B) by HCS assay.

FIG. 6 shows the result of FACS affinity test on NCI-H1650 cells.

FIG. 7 shows the result of Domain determination test, with ELISA binding on huCDH3 ECD domain1 (A), domain1+2 (B), domain1+2+3 (C), domain1+2+3+4 (D), and ECD (E).

FIGS. 8A-8B show the body weight change (A) and tumor growth inhibition (B) results of Study I single dose in vivo efficacy test in xenografted HCC70 breast tumor model.

FIGS. 9A-9B show the body weight change (A) and tumor growth inhibition (B) results of Study II dose response in vivo efficacy test in xenografted HCC70 breast tumor model.

FIGS. 10A-10B show the body weight change (A) and tumor growth inhibition (B) results of Study II dose response in vivo efficacy test in xenografted NCI-H1650 lung cancer model.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. All publications, patent applications, patents, and other references mentioned herein are incorporated by reference in their entirety.

### Definitions

In order to better understand the disclosure, the definitions and explanations of the relevant terms are provided as follows.

The term “antibody” or “Ab,” as used herein, generally refers to a Y-shaped tetrameric protein comprising two heavy (H) and two light (L) polypeptide chains held together by covalent disulfide bonds and non-covalent interactions. Light chains of an antibody may be classified into k and A light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (CL). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains generally follows the definition in Kabat Sequences of Proteins of Immunological Interest (National Institutes of Health, Bethesda, Md. (1987 and 1991)), Chothia & Lesk (1987) J. Mol. Biol. 196:901-917; Chothia et al., (1989) Nature 342:878-883; and/or IMGT (http://www.imut.org/). Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen binding fragments of an antibody may be produced by recombinant DNA techniques or by enzymatic or chemical cleavage of an intact antibody. Under some conditions, antigen binding fragments include Fab, Fab′, F(ab′)2, Fd, Fv, dAb and complementary determining region (CDR) fragments, single chain antibody (e.g. scFv), chimeric antibody, diabody and such polypeptides that comprise at least part of antibody sufficient to confer the specific antigen binding ability on the polypeptides.

Antigen binding fragments of an antibody may be obtained from a given antibody (e.g., the monoclonal anti-human P-cadherin antibody provided in the instant application) by conventional techniques known by a person skilled in the art (e.g., recombinant DNA technique or enzymatic or chemical cleavage methods), and may be screened for specificity in the same manner by which intact antibodies are screened.

“Fc” with regard to an antibody refers to that portion of the antibody comprising the second and third constant regions of a first heavy chain bound to the second and third constant regions of a second heavy chain via disulfide bonding, optionally also comprising part or whole of the hinge region. The Fc portion of the antibody is responsible for various effector functions such as antibody-dependent cell-mediated cytotoxicity (ADCC), and complement dependent cytotoxicity (CDC), but does not function in antigen binding.

The term “P-cadherin”, as used herein, refers to placental Cadherin and is a member of the classical cadherin family of transmembrane glycoproteins that regulate cell-cell adhesion. The exemplary sequences of human P-cadherin (encoded by CDH3 gene) can be obtained from Uniprot database under ID P22223, including a canonical sequence and several isoforms. The term “P-cadherin” herein is intended to include human, mouse, cyno P-cadherin, splice/allelic variants and fragments/derivatives thereof, and recombinant chimeric forms of P-cadherin, which can be prepared by standard recombinant expression methods or purchased commercially. The canonical P-cadherin sequence comprises 829 amino acids, wherein the mature protein begins at amino acid 108 with three distinct domains: five extracellular cadherin repeats (548 aa), single transmembrane region (23 aa) and highly conserved cytoplasmic tail (151 aa).

The terms “E-cadherin” and “N-cadherin”, as used herein, refers to epithelial Cadherin and neural cadherin, respectively, which are also members of the classical cadherin family. Cadherins are divided into type I and type II subgroups. Type I cadherins include E-cadherin, N-cadherin, P-cadherin and retinal cadherin (R-cadherin), whereas kidney cadherin (K-cadherin) and osteoblast cadherin (OB-cadherin) are type II cadherins. E-cadherin is encoded by CDH1 gene in human, which shares 66% homology with CDH3 gene. N-cadherin is encoded by CDH2 gene in human. E-cadherin, N-cadherin and P-cadherin are the best characterized subgroup of adhesion proteins.

The term “anti-P-cadherin antibody” or “P-cadherin antibody” or “antibody against P-cadherin,” as used herein, refers to an antibody, as defined herein, capable of binding to a P-cadherin, for example, binding to the ECD region of a human P-cadherin protein.

The term “monoclonal antibody” or “mAb,” as used herein, refer to a preparation of antibody molecules of single molecular composition. A monoclonal antibody displays a binding specificity and affinity for a particular antigen.

The term “fully human” as used herein, with reference to antibody or antigen-binding domain, means that the antibody or the antigen-binding domain has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “ADC” or “antibody-drug conjugate” or “immunoconjugate” can be used interchangeably herein, and comprises an antibody conjugated to a drug moiety, such as a cytotoxic or cytostatic agent e.g. a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate). An ADC generally has the formula Ab-(L-D)p, wherein Ab is the antibody or antigen-binding portion thereof, L is a linker system, D is the drug moiety, and p is a integer from 1 to 20.

The term “DAR” or “Drug-to-Antibody Ratio”, as used herein, refers to the average number of drugs conjugated to an antibody, which is an important attribute of ADCs. The DAR value affects the efficacy of the drug, as low drug loading reduces the potency, while high drug loading can negatively affect pharmacokinetics (PK) and toxicity. Various analytical methods can be used to measure DAR, such as Ultraviolet-Visible (UV/Vis) spectroscopy, Hydrophobic interaction chromatography (HIC), Reversed phase high-performance liquid chromatography (RP-HPLC) and Liquid chromatography coupled with electrospray ionization mass spectrometry (LC-ESI-MS).

Hydrophobic interaction chromatography (HIC) is a leading technique for the characterization of DAR values and drug loading distribution. The conjugated species are separated based on an increased hydrophobicity caused by the increased drug-load. In terms of cysteine-conjugated ADCs, the unconjugated antibody with the least hydrophobicity is eluted first while the most hydrophobic, most drug conjugated form elutes last, generating a quantitative elution profile. The area percentage of a peak represents the relative amount of each drug-loaded ADC species. The payload distribution is derived from the HIC profile while the average DAR is also calculated from the percentage peak area. As demonstrated herein, the DAR of the anti-P-cadherin ADCs as disclosed herein is in a range of about 1 to about 8. In some embodiment as demonstrated herein, the DAR of the anti-P-cadherin ADCs as disclosed herein is about 1 or 2 or 3 or 4 or 5 or 6 or 7 or 8. In some embodiment as demonstrated herein, the DAR of the anti-P-cadherin ADCs as disclosed herein is about 4.

The term “payload distribution”, as used herein, is another quality attribute of an ADC and is determined by fractionation of antibodies containing different number of drugs. DAR and payload distribution are not only the measurements of ADC product homogeneity but they also determine the amount of payload delivered to the target tissues, directly affecting both ADC efficacy and safety. Besides, DAR and payload distribution assessments are both important quality control criteria in ADC manufacturing.

The term “free payload control”, as used herein, refers to a linker linked to a drug moiety without loading the antibody. For example, for a ADC with the formula Ab-(L-D)p, free payload control refers to L-D.

The term “cytotoxic activity”, as used herein, refers to a cell-killing, cytostatic or growth inhibitory effect of an antibody-drug conjugate or an intracellular metabolite of an antibody-drug conjugate. Cytotoxic activity may be expressed as the IC₅₀ value, which is the concentration (molar or mass) per unit volume at which half the cells survive. The ADCs as disclosed herein are demonstrated to have a killing effect on human P-cadherin expressing cancer cells with an IC50 of no more than 0.1 nM, e.g. no more than 0.09 nM, no more than 0.08 nM, no more than 0.07 nM, no more than 0.06 nM, no more than 0.05 nM, no more than 0.04 nM, no more than 0.03 nM, no more than 0.02 nM or even less.

The term “linker”, as used herein, refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙ O(CR₂)ₙ—, repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide.

The term “KD” as used herein, is intended to refer to the equilibrium dissociation constant of a particular antibody (or ADC)-antigen interaction, which is obtained from the ratio of Koff to Kon and is expressed as a molar concentration (M). The term “Kon,” as used herein, is intended to refer to the association rate constant of a particular antibody-antigen interaction, whereas the term “Koff” as used herein, is intended to refer to the dissociation rate constant of a particular antibody-antigen interaction. KD values for antibodies can be determined using methods well established in the art.

The term “high affinity” as used herein, refers to the strength of the binding interaction between antigen and antibody (or ADC). Various methods are established in the art for measuring affinity, such as surface plasmon resonance, FACS affinity test, FACS binding test, and ELISA binding. In some embodiments, the ADC as disclosed herein has a K_{D} of 1×10⁻⁹ M or less, more preferably 5×10⁻¹⁰ M or less, more preferably 4×10⁻¹⁰ M or less, more preferably 3×10⁻¹⁰ M or less, more preferably 2×10⁻¹⁰ M or less, even more preferably 1×10⁻¹⁰ M or less for a target antigen expressed on cell surface, for example, on P-cadherin expressing cells, as measured by FACS affinity test.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM” or “M”. In some embodiments, the ADC as disclosed herein has a binding affinity at 1 nM or less, more preferably 0.5 nM or less, more preferably 0.1 nM or less, with P-cadherin expressing cells.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a P-cadherin protein is substantially free of antibodies that specifically bind antigens other than P-cadherin proteins). An isolated antibody that specifically binds a human P-cadherin protein may, however, have cross-reactivity to other antigens, such as P-cadherin proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human P-cadherin gene was transfected into 293F cells.

The term “hybridoma” and the term “hybridoma cell line,” as used herein, may be used interchangeably. When the term “hybridoma” and the term “hybridoma cell line” are mentioned, they also include subclone and progeny cell of hybridoma.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of P-cadherin-related diseases or conditions, refers to the ADC as disclosed herein in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro AR, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### Anti-P-Cadherin Antibodies

In some aspects, the disclosure provides an isolated antibody or an antigen-binding portion thereof against P-cadherin.

In some embodiments, the antibody or antigen-binding portion thereof comprises:
(A) a heavy chain variable region comprising SEQ ID NO: 7 or an amino acid sequence with at least 85%, at least 90%, at least 91%, at least 92%, at least 93%, at least 94%, at least 95%, at least 96%, at least 97%, at least 98%, or at least 99% sequence identity to SEQ ID NO: 7, and/or
(B) a light chain variable region comprising SEQ ID NO: 8 or an amino acid sequence with at least 85%, at least 90%, at least 91%, at least 92%, at least 93%, at least 94%, at least 95%, at least 96%, at least 97%, at least 98%, or at least 99% sequence identity to SEQ ID NO: 8.

In some embodiments, the isolated antibody or antigen-binding portion thereof comprises a heavy chain variable region (V_{H}) and a light chain variable region (V_{L}), wherein: the V_{H} comprises one or more heavy chain CDRs (HCDRs) selected from the group consisting of:
(i) a HCDR1 comprising the amino acid sequence of SEQ ID NO: 1 or an amino acid sequence that differs from SEQ ID NO: 1 by an amino acid addition, deletion or substitution of not more than 1, 2 or 3 amino acids;
(ii) a HCDR2 comprising the amino acid sequence of SEQ ID NO: 2 or an amino acid sequence that differs from SEQ ID NO: 2 by an amino acid addition, deletion or substitution of not more than 1, 2 or 3 amino acids; and
(iii) a HCDR3 comprising the amino acid sequence of SEQ ID NO: 3 or an amino acid sequence that differs from SEQ ID NO: 3 by an amino acid addition, deletion or substitution of not more than 1, 2 or 3 amino acids; and

the V_{L} comprises one or more light chain CDRs (LCDRs) selected from the group consisting of:
(i) a LCDR1 comprising the amino acid sequence of SEQ ID NO: 4 or an amino acid sequence that differs from SEQ ID NO: 4 by an amino acid addition, deletion or substitution of not more than 1, 2 or 3 amino acids;
(ii) a LCDR2 comprising the amino acid sequence of SEQ ID NO: 5 or an amino acid sequence that differs from SEQ ID NO: 5 by an amino acid addition, deletion or substitution of not more than 1, 2 or 3 amino acids; and
(iii) a LCDR3 comprising the amino acid sequence of SEQ ID NO: 6 or an amino acid sequence that differs from SEQ ID NO: 6 by an amino acid addition, deletion or substitution of not more than 1, 2 or 3 amino acids.

In some embodiments, the isolated antibody or antigen-binding portion thereof comprises a HCDR1 comprising the amino acid sequence of SEQ ID NO: 1; a HCDR2 comprising the amino acid sequence of SEQ ID NO: 2; a HCDR3 comprising the amino acid sequence of SEQ ID NO: 3; a LCDR1 comprising the amino acid sequence of SEQ ID NO: 4; a LCDR2 comprising the amino acid sequence of SEQ ID NO: 5; a LCDR3 comprising the amino acid sequence of SEQ ID NO: 6.

In some embodiments, the V_{H} or V_{L} region comprises an amino acid sequence with addition, deletion and/or substitution of one or more (e.g. 2, 3, 4, 5, 6, 7, 8, 9, 10) amino acids compared with the amino acid sequence as set forth in SEQ ID NO: 7 or 8, respectively. In some embodiments, the addition, deletion and/or substitution of at least one of the amino acids in the V_{H} or V_{L} region is not in any of the CDR sequences, but in the framework (FRW) sequences. For example, the isolated antibody or antigen-binding portion thereof as described above may comprise one or more substitutions of the amino acids in the framework sequences, e.g. FRW1, FRW2, FRW3, and/or FRW4 of the V_{H} or V_{L} region.

In certain embodiments, the isolated antibody or antigen-binding portion thereof as provided herein comprise any suitable framework region (FR) sequences, as long as the antigen-binding domains can specifically bind to P-cadherin.

In certain embodiments, the above described antibody is a monoclonal antibody. In some embodiments, the antigen-binding portion is an antibody fragment selected from a Fab, Fab′-SH, Fv, scFv, or (Fab′)2 fragment. In some embodiments, the antibody is humanized. In some embodiments, the antibody is fully human.

### Antibody-Drug Conjugates (ADCs)

In some aspects, the disclosure provides immunoconjugates or antibody-drug conjugates comprising the above described antibodies conjugated to a cytotoxic or cytostatic agent. The cytotoxic agent may include but not limited to, a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The term “conjugate” is used herein with its broadest definition to mean joined or linked (such as convalently linked) together. Molecules are “conjugated” when they act or operate as if joined.

The use of antibody-drug conjugates for local delivery of cytotoxic or cytostatic agents, i.e. drugs to kill or inhibit tumor cells in the treatment of cancer allows targeted delivery of the drug moiety to tumors, and intracellular accumulation therein, where systemic administration of unconjugated drug agents may result in unacceptable levels of toxicity to normal cells as well as the tumor cells sought to be eliminated (Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Both polyclonal antibodies and monoclonal antibodies have been reported as useful in these strategies (Rowland et al., (1986) Cancer Immunol. Immunother., 21:183-87). Drugs that can be used in ADCs include chemotherapeutic agents such as daunomycin, doxorubicin, methotrexate, and vindesine; toxins, for example bacterial toxins such as diphtheria toxin, plant toxins such as ricin, small molecule toxins such as geldanamycin, maytansinoids, and calicheamicin; auristatin peptides, auristatin E (AE) and monomethylauristatin (MMAE), which are synthetic analogs of dolastatin. MMAE is a synthetic derivative of dolastatin 10, a natural cytostatic pseudo peptide. The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ADCs for treatment of various cancers have been developed, such as MYLOTARG (gemtuzumab ozogamicin, an antibody drug conjugate composed of an anti-CD33 antibody linked to calicheamicin), Adcetris (bretuximab vedotin, which links an anti-CD30 antibody to MMAE), Kadcyla (ado-trastuzumab emtansine; T-DM1), SGN-CD33A (vadastuximab talirine), Rova-T (rovalpituzumab tesirine), and BAT8001 (a humanized anti-HER2 antibody covalently linked to maytansine derivative via a stable linker).

Chemotherapeutic agents useful in the generation of immunoconjugates are described herein below.

Enzymatically active toxins and fragments thereof that can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993. A variety of radionuclides are available for the production of radioconjugated antibodies. Examples include 212Bi, 131I, 131In, 90Y, and 186Re.

Conjugates of the anti-P-cadherin antibody and one or more small molecule toxins, such as a calicheamicin, maytansinoids, dolastatins, auristatins, a trichothecene, and CC1065, and the derivatives of these toxins that have toxin activity, are also contemplated herein.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987) Science, 238:1098. Carbon-14-labeled 1-isothiocyanatobenzyl-3-methyldiethylene triaminepentaacetic acid (MX-DTPA) is an exemplary chelating agent for conjugation of radionucleotide to the antibody (WO94/11026).

In certain embodiments, the disclosure provides an anti-P-cadherin monoclonal antibody conjugated to a cytotoxic agent, by linking W3195-pl via a protease-cleavable linker to the cytotoxic agent. In some embodiments, the cytotoxic agent is MMAE. Specifically, the linker system in such ADCs may comprise a thiol reactive maleimidocaproyl spacer, a dipeptide valine-citrulline linker (“vc”), and a self-immolative, p-amino-benzyloxycarbonyl (“PAB”), which is designed to be stable in the bloodstream. Binding to P-cadherin on the cell surface initiates internalization. Upon internalization into P-cadherin-expressing tumor cells, MMAE, which exerts its potent cytostatic effect by inhibiting microtubule assembly, tubulin-dependent GTP hydrolysis and polymerization, is released via proteolytic cleavage. Finally, after binding to tubulin, MMAE disrupts the microtubule network within the cell, which, in turn, induces cell cycle arrest and results in apoptotic death of the P-cadherin-expressing tumor cells.

### Maytansine and Maytansinoids

In some embodiments, the immunoconjugate comprises the antibody or antigen-binding portion thereof as disclosed herein conjugated to one or more maytansinoid molecules.

Maytansinoids are mitototic inhibitors which act by inhibiting tubulin polymerization. Maytansine was first isolated from the east African shrub Maytenus serrata and later discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230 and 4,371,533.

Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Maytansine compounds suitable for use as maytansinoid drug moieties are well known in the art and can be isolated from natural sources according to known methods, produced using genetic engineering techniques (see Yu et al (2002) PNAS 99:7968-7973), or maytansinol and maytansinol analogues prepared synthetically according to known methods. Suitable maytansinoids are disclosed, for example, in U.S. Pat. No. 5,208,020. Preferred maytansinoids are maytansinol and maytansinol analogues modified in the aromatic ring or at other positions of the maytansinol molecule, such as various maytansinol esters.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H₂S or P2S₅); C-14-alkoxymethyl(demethoxy/CH₂ OR)(U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Exemplary embodiments of maytansinoid drug moieties include: DM1, DM3 and DM4. The structure of DM1 is shown below:

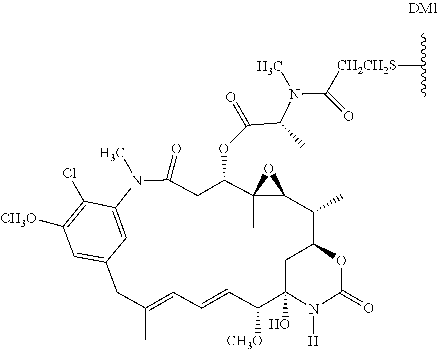

wherein the wavy line indicates the covalent attachment of the sulfur atom of the drug to a linker (L) of an antibody drug conjugate. HERCEPTIN (trastuzumab) linked by SMCC to DM1 has been reported (WO 2005/037992, which is expressly incorporated herein by reference in its entirety).

An Exemplary maytansinoid antibody drug conjugate “Ab-(SMCC-DM1)p” with the following structure and abbreviation (wherein Ab is antibody and p is 1 to about 8) is shown below (e.g. linked via SMCC):

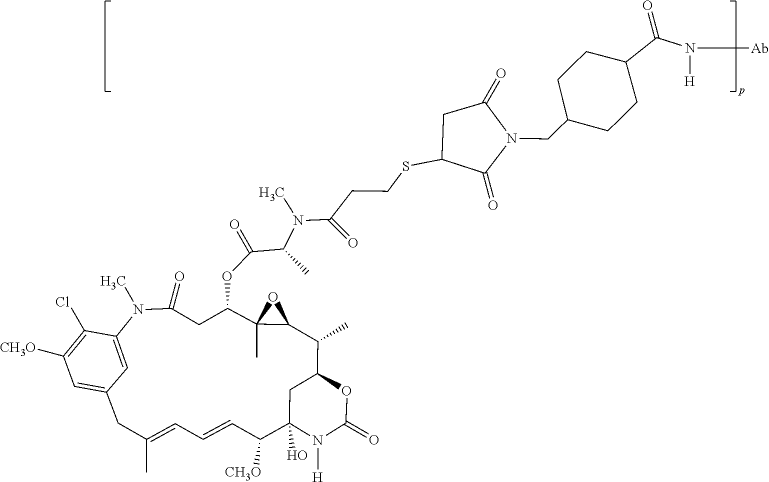

Anti-P-Cadherin antibody-maytansinoid conjugates can be prepared by chemically linking the antibody or antigen-binding portion thereof to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody.

There are many linking groups known in the art for making antibody-maytansinoid conjugates, including, for example, those disclosed in U.S. Pat. Nos. 5,208,020, 6,441,163, or EP Patent 0 425 235 B1, Chari et al., Cancer Research 52:127-131 (1992), and US 2005/0169933 A1, the disclosures of which are hereby expressly incorporated by reference. Antibody-maytansinoid conjugates comprising the linker component SMCC may be prepared as disclosed in U.S. patent application Ser. No. 11/141,344. The linking groups include disulfide groups, thioether groups, acid labile groups, photolabile groups, peptidase labile groups, or esterase labile groups, as disclosed in the above-identified patents. Additional linking groups are described and exemplified herein.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

In some embodiments, the antibodies as disclosed herein (full length or fragment) are conjugated to one or more maytansinoid molecules. In some embodiments of the immunoconjugate, the cytotoxic agent is a maytansinoid DM1. In some embodiments of the immunoconjugate, the linker is selected from the group consisting of SPDP, SMCC, IT, SPDP, and SPP. Some exemplary maytansinoid antibody drug conjugates may be Ab-(SPP-DM1)p, Ab-(SMCC-DM1)p, Ab-(BMPEO-DM1)p.

Immunoconjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference.

### Auristatins and Dolostatins

In some embodiments, the immunoconjugate comprises the anti-P-cadherin antibody disclosed herein conjugated to dolastatins or dolostatin peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division and have anticancer and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF, disclosed in “Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE (wherein the wavy line indicates the covalent attachment to a linker (L) of an antibody drug conjugate):

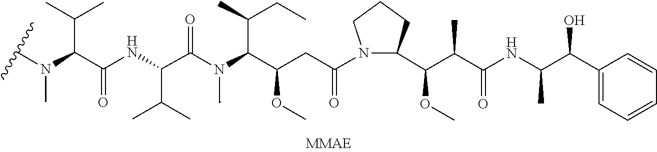 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Another exemplary auristatin embodiment is MMAF (wherein the wavy line indicates the covalent attachment to a linker (L) of an antibody drug conjugate):

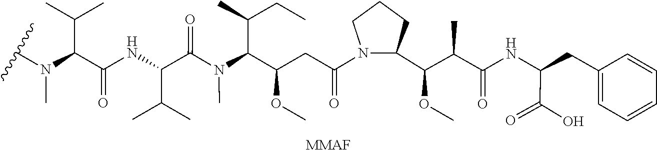

Additional exemplary embodiments comprising MMAE or MMAF and various linker components have the following structures and abbreviations (wherein Ab means antibody and p is 1 to about 8) are shown below. For example, VcMMAE (Mc-vc-PAB-MMAE) is obtained by using MMAE linked via p-aminobenzyloxycarbonyl (“PAB”) to the lysosomally cleavable dipeptide valine-citrulline (vc) and a thiolreactive maleimidocaproyl spacer (MC).

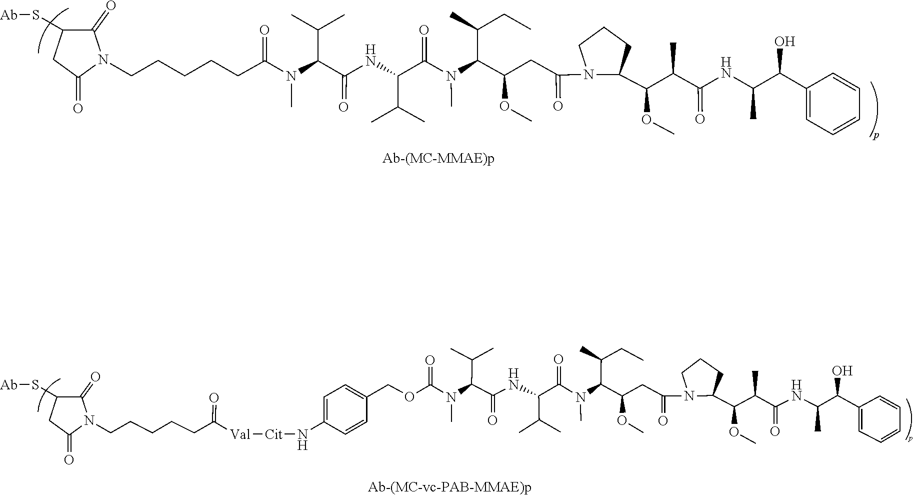

Typically, peptide-based drug moieties can be prepared by forming a peptide bond between two or more amino acids and/or peptide fragments. Such peptide bonds can be prepared, for example, according to the liquid phase synthesis method (see E. Schröder and K. Lübke, “The Peptides”, volume 1, pp 76-136, 1965, Academic Press) that is well known in the field of peptide chemistry. The auristatin/dolastatin drug moieties may be prepared according to the methods of Doronina (2003) Nat Biotechnol 21(7):778-784.

### Calicheamicin

In some other embodiments, the immunoconjugate comprises an antibody or antigen-binding portion thereof conjugated to one or more calicheamicin molecules. The calicheamicin family of antibiotics are capable of producing double-stranded DNA breaks at sub-picomolar concentrations. Structural analogues of calicheamicin which may be used include, but are not limited to, γ₁^{I}, α₂^{I}, α₃^{I}, N-acetyl-γ₁^{I}, PSAG and θ₁^{I}. Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

### Other Cytotoxic Agents

Other antitumor agents that can be conjugated to the antibodies as disclosed herein include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of chemotherapeutic agents that may be conjugated with antibodies the present disclosure include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK R polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

The present disclosure further contemplates an immunoconjugate formed between an antibody and a compound with nucleolytic activity (e.g., a ribonuclease or a DNA endonuclease such as a deoxyribonuclease; DNase).

For selective destruction of the tumor, the ADC may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the cytotoxic drug in the cell. For example, an acid-labile linker, peptidase-sensitive linker, photolabile linker, dimethyl linker or disulfide-containing linker may be used.

### Drug Loading

Drug loading is represented by p and DAR, DAR is the average number of drug moieties per antibody in a molecule of common ADC Formula Ab-(L-D)p, wherein Ab refers to the anti-P-cadherin antibody or antigen-binding portion thereof as disclosed herein, L refers to the linker and D refers to the drug moiety, generally comprising a cytotoxic or cytostatic agent, and p refers to the integer number of drug moieties for Ab molecule. Drug loading may range from 1 to 20 drug moieties (D) per antibody. ADCs include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis. For example, the W3195-p1-MMAE conjugate as disclosed herein has a DAR of around 4, as determined by hydrophobic interaction chromatography (HIC).

For some antibody-drug conjugates, p may be limited by the number of attachment sites on the antibody. For example, where the attachment is a cysteine thiol, an antibody may have only one or several cysteine thiol groups, or may have only one or several sufficiently reactive thiol groups through which a linker may be attached. In some instances, higher drug loading, e.g. p >5, may cause aggregation, insolubility, toxicity, or loss of cellular permeability of certain antibody-drug conjugates. In certain embodiments, the drug loading for an ADC of the disclosure ranges from 1 to about 8; from about 2 to about 6; from about 3 to about 5; from about 3 to about 4; from about 3.5 to about 4.5, from about 3.6 to about 4.4, from about 3.7 to about 4.3, from about 3.8 to about 4.2, or from about 3.9 to about 4.1. In some embodiments, the ADC ranges from about 3.5 to about 4.5. Indeed, it has been shown that for certain ADCs, the optimal ratio of drug moieties per antibody may be less than 8, and may be about 2 to about 5. See US 2005-0238649 A1 (herein incorporated by reference in its entirety).

In certain embodiments, fewer than the theoretical maximum of drug moieties are conjugated to an antibody during a conjugation reaction. An antibody may contain, for example, lysine residues that do not react with the drug-linker intermediate or linker reagent. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, the antibody as disclosed herein is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, March 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### Preparation of Antibody Drug Conjugates

In the antibody drug conjugates (ADC) as disclosed herein, the anti-P-cadherin antibody (Ab) or antigen-binding portion thereof is conjugated to one or more drug moieties (D), e.g. about 1 to about 20 drug moieties per antibody, about 1 to about 10 drug moieties per antibody, about 1 to about 8 drug moieties per antibody, about 1 to about 5 drug moieties per antibody, about 1 to about 4 drug moieties per antibody, about 1 to about 3 drug moieties per antibody, or about 1 to about 2 drug moieties per antibody, through a linker (L). In some embodiments, the number of drug moieties (D) per antibody is from about 1 to about 5, from about 2 to about 6, from about 2 to about 5, or from about 3 to about 4 drug moieties per antibody. Because the number of drug moieties per antibody is typically an average number over all conjugates in a population of an antibody drug conjugate, the number of drug moieties per antibody may not be a whole number.

The ADCs may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. In some embodiments, the MMAE drug moiety linked with MC-VC-PAB (i.e. MC-VC-PAB-MMAE) is commercially available (Lenena, biopharma) and can be directly used for conjugation with the antibody.

As described above, the linker may be composed of one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (“PAB”), N-Succinimidyl 4-(2-pyridylthio) pentanoate (“SPP”), N-Succinimidyl 4-(N-maleimidomethyl) cyclohexane-1 carboxylate (“SMCC′), and N-Succinimidyl (4-iodo-acetyl) aminobenzoate (“SIAB”). In some embodiments, the linker is MC-vc-PAB. Additional linker components are known in the art and some are described herein.

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzyme, for example, a tumor-associated protease, cathepsin B, C and D, or a plasmin protease.

Nucleophilic groups on antibodies include but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as TCEP. Each cysteine bridge will thus form, theoretically, two reactive thiol nucleophiles.

Antibody drug conjugates of the disclosure may also be produced by modification of the antibody to introduce electrophilic moieties, which can react with nucleophilic substituents on the linker reagent or drug.

Methods for the conjugation of linker-drug moieties to cell-targeted proteins such as antibodies, immunoglobulins or fragments thereof are found, for example, in WO2006/034488 (incorporated by reference herein). Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

As illustrated in the Examples, the anti-P-cadherin antibody as disclosed herein may be prepared by reducing with TCEP and then conjugating with a commercially purchased MC-vc-PAB-MMAE, i.e. linker already linked to the drug moiety.

Anti-P-Cadherin ADCs with Certain Properties

The ADCs of the present disclosure are characterized by particular functional features or properties. The in vitro and in vivo functional characteristics and pharmacological activity of the antibodies and ADCs have been fully assessed at the molecular and cellular levels according to the mechanism of action for the target. The ADCs as disclosed herein may have one or more of the following properties:
(a) bind to cell surface human P-cadherin expressing cells with an EC50 in nM grade (e.g. no more than 1 nM, no more than 0.5 nM, no more than 0.3 nM, no more than 0.2 nM, no more than 0.1 nM, no more than 0.09 nM, no more than 0.08 nM), as measured by FACS;
(b) have good internalization ability comparable with benchmark ADCs;
(c) being stable in serum for at least 14 days;
(d) show better cytotoxicity effect on human P-cadherin expressing cells compared with benchmark ADCs, and no killing effect on P-cadherin low expressing cells or normal cells;
(e) show better binding to human P-cadherin compared with benchmark ADCs, as measured by FACS affinity test;
(f) show no non-specific binding to human cell lines with low expression of P-cadherin, in contrast to benchmark ADC; and
(g) show significantly better tumor inhibition in in vivo tumor model compared with benchmark ADCs and a good dose-dependent antitumor effect.

### Pharmaceutical Compositions

In some aspects, the disclosure is directed to a pharmaceutical composition comprising the anti-P-cadherin ADCs as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-P-cadherin antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. In some embodiments, the present disclosure provides a composition comprising the ADCs as disclosed herein and one or more anti-oxidants such as methionine. The present disclosure further provides a variety of methods, wherein the ADC is mixed with one or more anti-oxidants, such as methionine, so that the ADCs can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the ADCs of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the ADCs of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the ADCs of the present disclosure will comprise multiple doses of the selected drug product over a period of weeks or months. More specifically, the ADCs of the present disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the ADCs as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the antibody or the antigen binding portion thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml

### Applications of the Disclosure

The ADCs, ADC compositions and methods of the present disclosure have numerous in vitro and in vivo utilities involving, for example, enhancement of immune response and targeted cytotoxicity effect. For example, these molecules can be administered to P-cadherin expressing cells in culture, in vitro or ex vivo, or to human subjects, e.g., in vivo, to kill or inhibit cell growth (e.g. cells with a high P-cadherin expression). The immune response against P-cadherin can also be modulated, for instance, augmented, stimulated or up-regulated.

In some embodiments, the subjects include human patients in need of enhancement of an immune response, such as a P-cadherin related immune response. In some embodiments, the subjects are in need of treatment of P-cadherin related cancers, such as cancers characterized by overexpression of P-cadherin. In some embodiments, the methods are particularly suitable for treatment of cancer in vivo, especially P-cadherin related cancers.

### Treatment of Disorders Including Cancers

In some aspects, the present disclosure provides a method of treating a disorder or a disease in a mammal, which comprises administering to the subject (for example, a human) in need of treatment a therapeutically effective amount of the ADCs as disclosed herein. The disorder or disease may be a cancer.

A variety of cancers where P-cadherin is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In some embodiments, the cancer is a P-cadherin positive solid tumor. In some specific embodiments, the cancer is breast cancer. In some other embodiments, the cancer is colorectal cancer, prostate cancer or NSCLC.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

The ADCs as disclosed herein may be used alone as a monotherapy, or may be used in combination with chemical therapies, radiotherapies, other targeted therapies or cell immunotherapies etc.

### Combination Therapy

The antibody-drug conjugate (ADC) as disclosed herein may be combined in a pharmaceutical combination formulation, or dosing regimen as combination therapy, with at least one additional compound having anti-cancer properties. The at least one additional compound of the pharmaceutical combination formulation or dosing regimen preferably has complementary activities to the ADC of the combination such that they do not adversely affect each other.

The at least one additional compound may be a chemotherapeutic agent, cytotoxic agent, cytokine, growth inhibitory agent, anti-hormonal agent, and/or cardioprotectant. Such molecules are suitably present in combination in amounts that are effective for the purpose intended. A pharmaceutical composition containing an ADC as disclosed herein may also have a therapeutically effective amount of a chemotherapeutic agent such as a tubulin-forming inhibitor, a topoisomerase inhibitor, or a DNA binder.

In some embodiments, the first compound is an anti-P-cadherin ADC of the disclosure and the at least one additional compound is a therapeutic antibody other than an anti-P-cadherin antibody or ADC). In some embodiments, the at least one additional compound is an anti-HER2 antibody, such as trastuzumab or pertuzumab. In some embodiments, the at least one additional compound is an antibody (either a naked antibody or an ADC) efficacious in treating a cell proliferative disease in a tissue expressing P-cadherin.

Other therapeutic regimens may be combined with the administration of an anticancer agent identified in accordance with this invention, including without limitation radiation therapy and/or bone marrow and peripheral blood transplants, and/or a cytotoxic agent, a chemotherapeutic agent, or a growth inhibitory agent. In one of such embodiments, a chemotherapeutic agent is an agent or a combination of agents such as, for example, cyclophosphamide, hydroxydaunorubicin, adriamycin, doxorubincin, vincristine (Oncovin™), prednisolone, CHOP, CVP, or COP, or immunotherapeutics such as anti-PSCA, anti-HER2 (e.g., Herceptin®, Omnitarg™) or anti-VEGF (e.g., Avastin®). The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

In one embodiment, treatment with an ADC involves the combined administration of an anticancer agent identified herein, and one or more chemotherapeutic agents or growth inhibitory agents, including coadministration of cocktails of different chemotherapeutic agents. Chemotherapeutic agents include taxanes (such as paclitaxel and docetaxel) and/or anthracycline antibiotics. Preparation and dosing schedules for such chemotherapeutic agents may be used according to manufacturer's instructions or as determined empirically by the skilled practitioner. Preparation and dosing schedules for such chemotherapy are also described in “Chemotherapy Service”, (1992) Ed., M. C. Perry, Williams & Wilkins, Baltimore, Md.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

### Pharmaceutical Packs and Kits

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds an antibody-drug conjugate (ADC) composition which is effective for treating the condition and may have a sterile access port (for example the container may be an intravenous solution bag or a vial having a stopper pierceable by a hypodermic injection needle). At least one active agent in the composition is an ADC. The label or package insert indicates that the composition is used for treating the condition of choice, such as cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Pharmaceutical packs and kits comprising one or more containers, comprising one or more doses of the ADC are also provided. In certain embodiments, a unit dosage is provided wherein the unit dosage contains a predetermined amount of a composition comprising, for example, the ADC with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the ADC of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the ADC and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Abbreviations

MC=6-maleimidocaproyl

Val-Cit or “vc”=valine-citrulline (an exemplary dipeptide in a protease cleavable linker)

PAB=p-aminobenzyloxycarbonyl (an example of linker component)

SPP=N-succinimidyl-4-(2-pyridylthio)pentanoate

SPDP=N-succinimidyl-3-(2-pyridyldithio) propionate

SMCC=succinimidyl-4-(N-maleimidomethyl) cyclohexane-1-carboxylate

MMAE=mono-methyl auristatin E (MW 718)

MMAF=variant of auristatin E (MMAE) with a phenylalanine at the C-terminus of the drug (MW 731.5)

DM1=N(2′)-deacetyl-N(2′)-(3-mercapto-1-oxopropyl)-maytansine

DM3=N(2′)-deacetyl-N2-(4-mercapto-1-oxopentyl)-maytansine

DM4=N(2′)-deacetyl-N2-(4-mercapto-4-methyl-1-oxopentyl)-maytansine

### Sequence Listing Summary

The sequences of CDRs, variable regions, constant regions of W3195-1.53.1-p1-uIgG1L are listed in the tables below.

TABLE A
Amino acid sequences of the variable region

| | | | |
|---|---|---|---|
| W3195-1.53.1-p1- | HCDR1 | HCDR2 | HCDR3 |
| uIgG1L | SEQ ID NO: 1 | SEQ ID NO: 2 | SEQ ID NO: 3 |
| | GGSVISDNYYWT | YIYYRGSTNYNPSLKN | DRRTGNSLPFDN |
| | LCDR1 | LCDR2 | LCDR3 |
| | SEQ ID NO: 4 | SEQ ID NO: 5 | SEQ ID NO: 6 |
| | SGDKLGDKFAS | QDNKRPS | QAWDSSIVV |

TABLE B
Amino acid sequences of the variable regions and constant regions of W3195-
1.53.1-p1-uIgG1L

| | | |
|---|---|---|
| VH | SEQ ID NO: 7 | QVQLQESGPGLVKPSETLSLTCTVSGGSVISDNYY |
| | | WTWVRQPPGKGLEFIGYIYYRGSTNYNPSLKNRVI |
| | | ISIDTSRNQFSLDLNSVTAADTAVYYCARDRRTGN |
| | | SLPFDNWGQGTLVTVSS |
| VL | SEQ ID NO: 8 | SYELTQPPSVSVSPGQTAQITCSGDKLGDKFASWY |
| | | QQKSGQSPVVVIYQDNKRPSGFPERFSGSNSGNTA |
| | | TLTISGTQAMDEADYYCQAWDSSIVVFGGGTKLT |
| | | VL |
| Heavy chain | SEQ ID NO: 9 | ASTKGPSVFPLAPSSKSTSGGTAALGCLVKDYFPE |
| Constant region | | PVTVSWNSGALTSGVHTFPAVLQSSGLYSLSSVVT |
| | | VPSSSLGTQTYICNVNHKPSNTKVDKRVEPKSCDK |
| | | THTCPPCPAPELLGGPSVFLFPPKPKDTLMISRTPE |
| | | VTCVVVDVSHEDPEVKFNWYVDGVEVHNAKTKP |
| | | REEQYNSTYRVVSVLTVLHQDWLNGKEYKCKVS |
| | | NKALPAPIEKTISKAKGQPREPQVYTLPPSREEMTK |
| | | NQVSLTCLVKGFYPSDIAVEWESNGQPENNYKTT |
| | | PPVLDSDGSFFLYSKLTVDKSRWQQGNVFSCSVM |
| | | HEALHNHYTQKSLSLSPGK |
| Light chain | SEQ ID NO: 10 | GQPKAAPSVTLFPPSSEELQANKATLVCLISDFYPG |
| Constant region | | AVTVAWKADSSPVKAGVETTTPSKQSNNKYAASS |
| | | YLSLTPEQWKSHRSYSCQVTHEGSTVEKTVAPTEC |
| | | S |

TABLE C
Amino acid sequences of the heavy chain and light chain of W3195-
1.53.1-p1-uIgG1L

| | |
|---|---|
| Heavy chain | SEQ ID NO: 11 |
| | QVQLQESGPGLVKPSETLSLTCTVSGGSVISDNYYWTWVRQPPGKGLE |
| | FIGYIYYRGSTNYNPSLKNRVIISIDTSRNQFSLDLNSVTAADTAVYYCA |
| | RDRRTGNSLPFDNWGQGTLVTVSSASTKGPSVFPLAPSSKSTSGGTAAL |
| | GCLVKDYFPEPVTVSWNSGALTSGVHTFPAVLQSSGLYSLSSVVTVPSS |
| | SLGTQTYICNVNHKPSNTKVDKRVEPKSCDKTHTCPPCPAPELLGGPSV |
| | FLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEK |
| | TISKAKGQPREPQVYTLPPSREEMTKNQVSLTCLVKGFYPSDIAVEWES |
| | NGQPENNYKTTPPVLDSDGSFFLYSKLTVDKSRWQQGNVFSCSVMHE |
| | ALHNHYTQKSLSLSPGK |
| Light chain | SEQ ID NO: 12 |
| | SYELTQPPSVSVSPGQTAQITCSGDKLGDKFASWYQQKSGQSPVVVIY |
| | QDNKRPSGFPERFSGSNSGNTATLTISGTQAMDEADYYCQAWDSSIVV |
| | FGGGTKLTVLGQPKAAPSVTLFPPSSEELQANKATLVCLISDFYPGAVT |
| | VAWKADSSPVKAGVETTTPSKQSNNKYAASSYLSLTPEQWKSHRSYS |
| | CQVTHEGSTVEKTVAPTECS |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Antigens, Benchmark Antibodies and Cell Lines

### 1.1 Preparation of Materials

Information on the commercially available materials used in the examples is provided in Table 1.

TABLE 1

| Materials | Vendor | Cat. |
|---|---|---|
| HCT-116 | ATCC | CCL-247 |
| HCC-1954 | ATCC | CRL-2338 |
| HCC-70 | ATCC | CRL-2315 |
| HT-29 | ATCC | HTB-38 |
| A549 | ATCC | CCL-185 |
| MDA-MB-453 | ATCC | HTB-131 |
| NCI-H1650 | ATCC | CRL-5883 |
| Jurkat | ATCC | TIB-152 |
| HepG2 | ATCC | HB-8065 |
| SK-OV-3 | ECACC | 91091004 |
| Calu-6 | ATCC | HTB-56 |
| A375 | ATCC | CRL-1619 |
| A204 | Jackson | HTB-82 |
| 8505C | Jackson | ACC 219 |
| H1 Hela | Promega | CRL-1958 |
| 293F | Thermo Fisher | R79007 |
| BT474 | ATCC | HTB-20 |
| Alexa647 conjugated Goat anti- | Jackson | 109-605-098 |
| human IgG Fc | | |
| PE conjugated Goat anti-human | Jackson | 109-115-098 |
| IgG Fc | | |
| Cell Titer Glo | Promega | G7573 |
| Fetal bovine serum (FBS) | Corning | 35-076-CV |
| Ni column | GE healthcare | 173712 |
| Protein A column | GE healthcare | 175438 |
| Protein G column | GE Healthcare | 170618 |
| HPLC-SEC | TOSOH | 0008541 |
| NuPAGE4%-12% Bis-Tris Gel | Thermo Fisher | NP0322BOX |

### 1.2 Construction of Expression Vector of Soluble Antigens

The amino acid sequence encoding the extracellular domain of human P-cadherin (Uniprot ID: P22223, aa 108-654) was first codon optimized for mammalian expression and then synthesized by GENEWIZ (SuZhou, CHINA). The DNA segment was then sub-cloned into the pcDNA3.3 expression vector with 6×His at the C-terminal. Protein samples of human, cyno and mouse P-cadherin were also purchased from Sino Biological.

### 1.3 Construction of Expression Vector of BMK Antibody

The amino acid sequences encoding the variable domain of two benchmark antibodies (WBP319-BMK4 as disclosed in WO2016075670A1, seq ID 8 and 18) were first codon optimized for mammalian expression and then synthesized by GENEWIZ (SuZhou, CHINA). The DNA segments were then sub-cloned into pcDNA3.4 expression vectors with constant region of human IgG1 or IgG4 (S228P).

### 1.4 Small Scale Expression of Protein

The plasmids containing VH and VL gene were co-transfected into Expi293 cells (Thermofisher, A14635). The cells were cultured for 5 days following the manufacturer suggested protocol. The supernatants were collected and analyzed by SDS-PAGE.

The plasmids containing VH and VL gene were co-transfected into ExpiCHO cells (Thermofisher, A29133). The cells were cultured for 10 days following the manufacturer suggested protocol. The supernatants were collected and analyzed by SDS-PAGE.

### 1.5 Purification of Fc-Tagged Protein

The supernatant of Expi293 cells or ExpiCHO cells-expressing target proteins was collected and filtered for purification using either Protein A column (GE Healthcare, Cat. 175438) or Protein G column (GE Healthcare, Cat. 170618). The concentration of purified Fc-tagged proteins was determined by absorbance at 280 nm. The size and purity were tested by SDS-PAGE and SEC-HPLC, respectively and then stored at −80° C.

### Example 2

Generation of Monoclonal Antibodies (mAbs) and Antibody-Drug Conjugates (ADCs)
2.1 Generation of W3195-1.53.1-p1-uIgG1L mAb The lead antibody, W3195-1.53.1-p1-uIgG1L (or abbreviated as W3195-p1), was obtained through immunizing transgenic OMT rats with human P-cadherin, hybridoma generation, a series of antibody screening and subcloning, and sequence optimization (e.g. PTM removal). The constant region is in wild-type human IgG1 format. The sequences of W3195-1.53.1-p1-uIgG1L are set forth in Tables A-C.
2.2 Generation of Benchmark ADC: WBP319-BMK4.uIgG1k-DM1

The antibody WBP319-BMK4.uIgG1k was dissolved in 50 mM PB, 50 mM NaCl, 2 mM EDTA, and pH 6.99 to 4.3 mg/mL. The organic co-solvent DMA (Alfa Aesar, A10924) was added to the antibody solution to a concentration of 20%, followed by 3.8 eq of SMCC-DM1 (Levenabiopharma, SET0101). Conjugation reaction was performed at 22° C. for 3 h. The conjugated product was purified with 30KD ultrafilter device (Millipore, UFC903024) and stored in 20 mM succinic acid, pH 5.0.

Final product was characterized with UV-vis for concentration and DAR, SEC-HPLC for DAR, aggregation and purity, RP-HPLC for free drug residue and Endosafe-PTS (Charlesriver, PTS100) for endotoxin.

2.3 Generation of W3195-1.53.1-p1-uIgG1L-MMAE (Also Named as W3195-p1-MMAE)

Antibody W3195-1.53.1-p1-uIgG1L was dissolved in 40 mM PB, 2 mM EDTA.Na2, pH 7.0 to a concentration of 3.5 mg/ml. 2.6 eq of TCEP (Pierce, 20490) was added to the antibody solution and the mixture was incubated at 37° C. for 2 h. Then DMA (Alfa Aesar, A10924) was added to the reduced antibody to a concentration of 10%, followed by 7 eq of MC-vc-PAB-MMAE (Levenabiopharma, SET0201). Conjugation reaction was performed at 4° ° C. for 1 h. The conjugated product was purified with 40KD MWCO desalting column (Zeba spin desalting column, Ref 87772) and stored in 20 mM Histidine-acetate pH5.5.

Final product was characterized with HIC-HPLC for DAR and drug distribution, SEC-HPLC for aggregation and purity, RP-HPLC for free drug residue and Endosafe-PTS (Charlesriver, PTS100) for endotoxin.

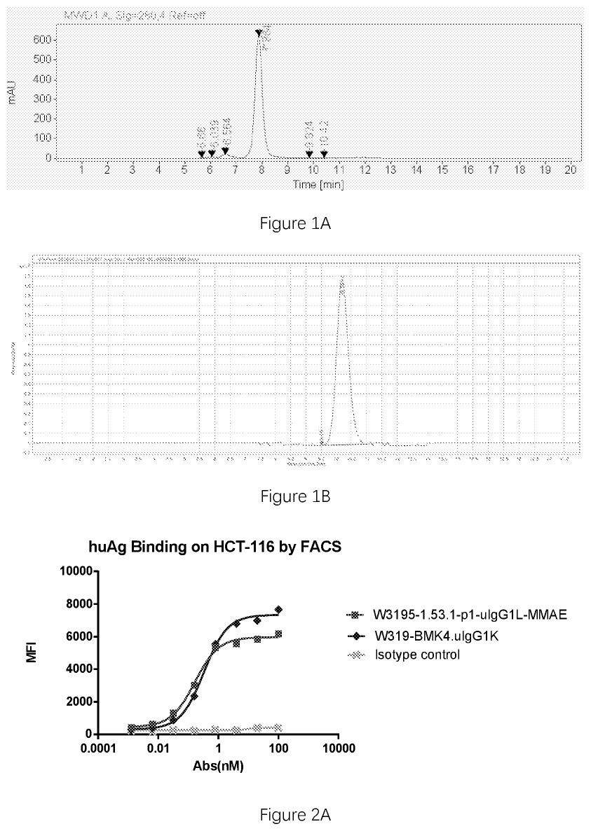

As shown in FIG. 1A-1B, the retention time of W3195-1.53.1-p1-uIgG1L-MMAE and WBP319-BMK4.uIgG1k-DM1 are at ˜7.864 mins and 10.19 mins, indicating both antibodies are normal. As shown in Tables 2A-B below, W3195-1.53.1-p1-uIgG1L succeeded in conjugation with MMAE, with DAR 3.86; WBP319-BMK4.uIgG1k succeeded in conjugation with DM1, with DAR 3.33.

TABLE 2A
Summary of W3195-1.53.1-p1-uIgG1L-MMAE characterizations

| | | HIC- | Purity | FD | Endotoxin | Conc. | Amount | |
|---|---|---|---|---|---|---|---|---|
| MMAE ADC | Lot# | DAR | (%) | (%) | ( [symbol] U/mg) | (mg/mL) | (mg) | Storage buffer |
| Criteria | NA | 4.00 | >95 | <5 | <0.5 | NA | NA | NA |
| W3195-1.53.1-p1- | wbp886-adc-4 | 3.86 | 96.06 | 0.54 | 0.014 | 3.05 | 21.35 | 20 mM Histidine: |
| uIgG1l-MMAE | | | | | | | | Acetate, pH 5.42 |

[symbol]

indicates data missing or illegible when filed

TABLE 2B
Summary of WBP319-BMK4.uIgG1k-DM1 characterizations

| | | Conc. | UV- | SEC- | Aggr | FD | Endotoxin | Amount | |
|---|---|---|---|---|---|---|---|---|---|
| DM1 ADC | Lot# | (mg/mL) | DAR | DAR | % | (%) | ( [symbol] U/mg) | (mg) | Storage buffer |
| WBP319- | wbp886- | 5.09 | 3.33 | 3.31 | 0.0 | 2.13 | 0.188 | ~56.0 | 20 mM succinate, |
| BMK4.uIgG1k-DM1 | 20190620-p | | | | | | | | PH 50 |

[symbol]

indicates data missing or illegible when filed

### Example 3

### In Vitro Characterization of ADC

### 3.1 Target Binding (FACS)

Binding of the ADCs to P-cadherin expressed on HCT-116 cells was determined by flow cytometry analysis using the same procedure as described above. In brief, HCT-116 or NCI-H1650 cells were harvested with Versene (Invitrogen, #15040066) and diluted to 1×10⁶ cells/ml in 1% BSA (Bovogen, #BSAS)/1×PBS (Ca+/Mg+) (Gibco, #14040-117). 1×10⁵ cells/well (100 μL) were added to each well of a 96-well U-plate (Corning, #3799) and centrifuged at 1500 rpm (Eppendorf, #5810R) for 5 minutes before removing the supernatant. Antibodies serially diluted in 1% BSA/1×PBS (Ca+/Mg+) were added at 100 μL/well to the pelleted cells and incubated at 4° C. for 1 hour. A non-related hlgG1 antibody was used as an isotype control. Cells were washed two times with 180 μL/well of 1% BSA/1×PBS (Ca+/Mg+) by centrifugation at 1500 rpm for 5 minutes at 4° C. Pelleted cells were resuspended in 100 μL/well Alexa647 conjugated Goat anti-human IgG Fc (Jackson, #109-605-098) 1:500 diluted in 1% BSA/1×PBS (Ca+/Mg+) for 30 minutes at 4° C. in the dark. Cells were then washed two times as described above. After the final wash, cells were resuspended in 100 μL 1% BSA/1×PBS (Ca+/Mg+) and fluorescence values were measured with a FACS Canto II cytometer (BD Biosciences). The amount of cell surface bound anti-P-Cadherin antibody was assessed by measuring the mean fluorescence (MFI). The FACS raw data were analyzed by FlowJo software, wells containing no antibody or secondary antibody only were used to establish background fluorescence. Binding EC50 values were obtained by the four-parameter non-linear regression analysis using GraphPad Prism6 software.

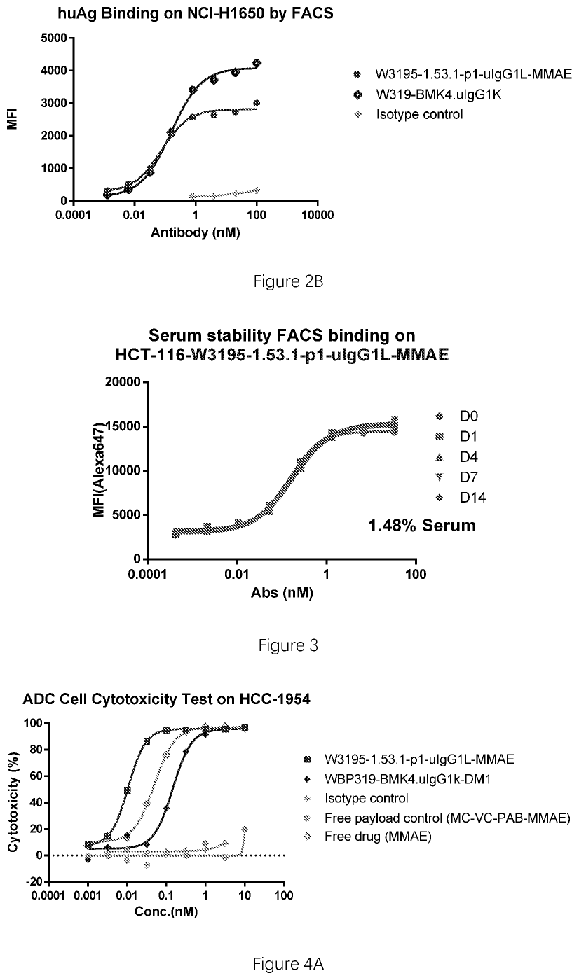

As shown in FIGS. 2A-2B and Table 3A-3B, W3195-1.53.1-p1-uIgG1L-MMAE showed binding to human P-cadherin-expressing HCT-116 cells with an EC50 of 0.17 nM, comparable with W319-BMK4.uIgG1K (0.33 nM); and showed binding to human P-cadherin-expressing NCI-H1650 cells with an EC50 of 0.075 nM, comparable with W319-BMK4.uIgG1K (0.16 nM).

Table 3A. FACS binding result of ADC to HCT-116 cells

HCT-116

| mAbs | EC50 (nM) | Max MFI |
|---|---|---|
| W3195-1.53.1-p1-uIgG1L-MMAE | 0.17 | 6164 |
| W319-BMK4.uIgG1K | 0.33 | 7651 |
| Isotype control | >100 | 402 |

Table 3B. FACS binding result of ADC to NCI-H1650 cells

NCI-H1650

| Abs | EC50 (nM) | Max MFI |
|---|---|---|
| W3195-1.53.1-p1-uIgG1L-MMAE | 0.075 | 3005 |
| W319-BMK4.uIgG1K | 0.16 | 4237 |
| Isotype control | NA | 331 |

### 3.2 Serum Stability

The antibody stability in human serum was tested by FACS. Briefly, human serum was freshly isolated by centrifuging the fresh blood twice at 4000 rpm for 10 minutes. Antibodies were mixed with freshly isolated human serum (serum content >95%) and incubated at 37° ° C. for 0, 1, 4, 7 and 14 days respectively, after which the samples were rapidly frozen in liquid nitrogen or dry ice/ethanol bath and stored at −80° C. until use. As a control, antibodies serum mix was frozen immediately without 37° C. incubation. For the FACS analysis, the samples from different time points were free-thawed simultaneously at 4° C. The thawed antibodies were serially diluted and added to 1×10⁵/well HCT-116 (ATCC, #CCL-247) cells and incubated for 1 hour at 4° C. The cells were washed twice with 1% BSA/1×PBS (Ca+/Mg+). Alexa647 conjugated Goat anti-human IgG Fc (Jackson, #109-605-098) 1:500 diluted in 1% BSA/1×PBS (Ca+/Mg+) were added to the cells and incubated at 4° C. for 30 minutes. Cells were washed twice in the same buffer and the mean fluorescence (MFI) of stained cells was measured using a FACS Canto II cytometer (BD Biosciences) and analyzed by FlowJo. Wells containing no antibody or secondary antibody only were used to establish background fluorescence. Four-parameter non-linear regression analysis was used to obtain EC50 values for cell binding using GraphPad Prism6 software.

As shown in FIG. 3 and Table 4, W3195-1.53.1-p1-uIgG1L-MMAE was stable in serum stability test for at least 14 days.

TABLE 4
FACS binding result with time

| | Day 0 | | Day 1 | | Day 4 | | Day 7 | | Day 14 | |
|---|---|---|---|---|---|---|---|---|---|---|
| | EC50 | Max | EC50 | Max | EC50 | Max | EC50 | Max | EC50 | Max |
| mAb/ADC | (nM) | MFI | (nM) | MFI | (nM) | MFI | (nM) | MFI | (nM) | MFI |
| W3195-1.53.1-p1-uIgG1L-MMAE | 0.14 | 14400 | 0.15 | 15100 | 0.18 | 15100 | 0.17 | 15700 | 0.18 | 15800 |

### 3.3 Cytotoxicity Assay of ADC

The ability of anti-P-Cadherin antibody drug conjugates to inhibit tumor cells growth was measured using in vitro cytotoxicity assays. HCC-1954 (ATCC, #CRL-2388), HCC-70 (ATCC, #CRL-2315), HT-29 (ATCC, #HTB-38), A549 (ATCC, #CCL-185), MDA-MB-453 (ATCC, #HTB-131) and NCI-H1650 cells were routinely cultured and assayed in RPMI1640 with 10% fetal bovine serum. For cell cytotoxicity assay, 50 μL of cells from each cell line were seeded onto 96-well clear bottom black plates (Greiner, #655090) so that the total cell number per well was at 4000 cells/well for HCC-1954, 6000 cells/well for HCC-70, 5000 cells/well for HT-29, 800 cells/well for A549, 5000 cells/well for MDA-MB-453 and 2000 cells/well for NCI-H1650. The cells were cultured overnight at 37° C. in a humidified 5% CO2 incubator before adding the appropriate concentration of anti-CDH3 antibody-drug conjugate, with the IgG1 isotype control antibody (50 μL/well). The plates were returned to the incubator for 5 days before cell viability assay using Cell Titer Glo (Promega, #G7573). 50 μL of Cell Titer Glo solution was added to each well and incubated at room temperature with gentle shaking for 10 minutes. The amount of luminescence was determined using determined using Envision (PerkinElmer). The extent of growth inhibition obtained with each antibody was calculated by comparing the luminscence values obtained with the control well without any antibody addition. The proliferation inhibition IC50 values of anti-P-Cadherin-ADCs were calculated by four-parameter non-linear regression analysis using GraphPad Prism6 software.

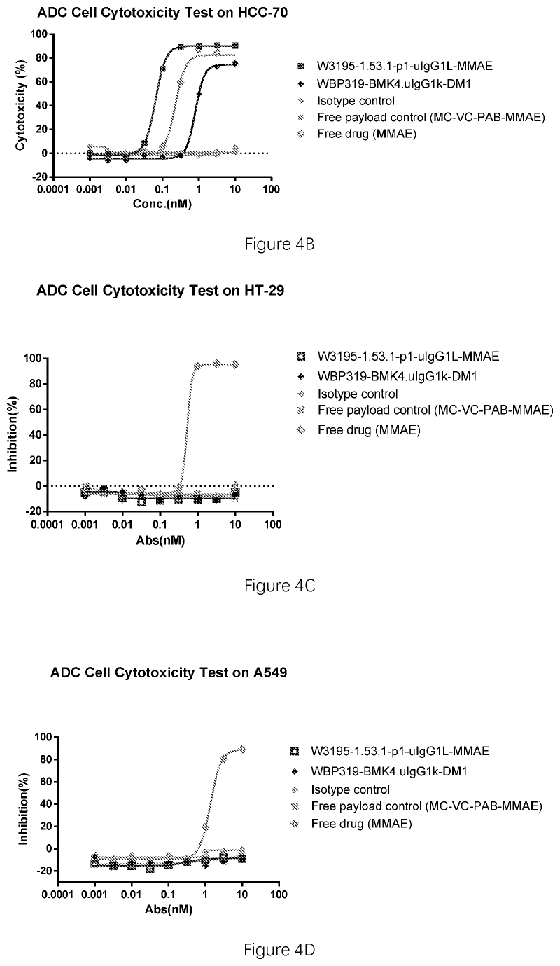

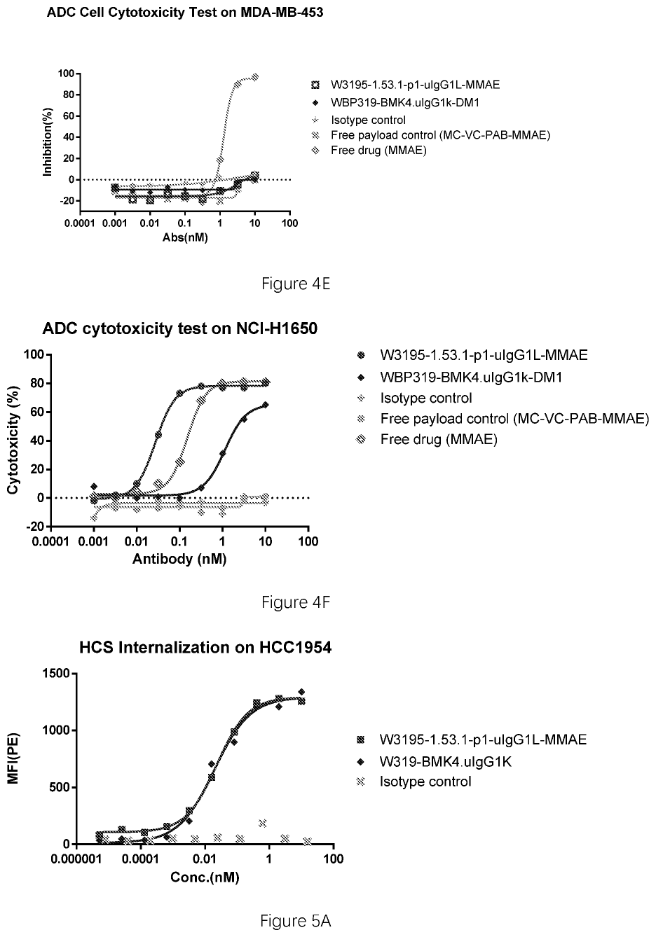

As shown in FIGS. 4A-4F and Tables 5A-F, W3195-1.53.1-p1-uIgG1L-MMAE showed good killing effect on human P-cadherin expressing HCC-1954 cells with an IC50 of 0.011 nM, better than WBP319-BMK4.uIgG1k-DM1 (0.15 nM); showed good killing effect on human P-cadherin expressing HCC-70 cells with an IC50 of 0.065 nM, better than WBP319-BMK4.uIgG1k-DM1 (0.80 nM); showed no killing effect on human P-cadherin low expressing HT-29 cells with an IC50>10 nM; showed no killing effect on human P-cadherin low expressing A549 cells with an IC50>10 nM; showed no killing effect on human P-cadherin low expressing MDA-MB-453 cells with an IC50>10 nM; and showed good killing effect on human P-cadherin expressing NCI-H1650 cells with an IC50 of 0.027 nM, better than WBP319-BMK4.uIgG1k-DM1 (1.14 nM).

TABLE 5A

| | ADC Cytotoxicity | |
|---|---|---|
| | on HCC-1954 | |
| Abs | IC50 (nM) | Max Inh % |
| W3195-1.53.1-p1-uIgG1L-MMAE | 0.011 | 96.9 |
| WBP319-BMK4.uIgG1k-DM1 | 0.15 | 96.8 |
| Isotype control | >10 | 9.3 |
| Free payload control (MC-VC-PAB-MMAE) | >10 | 20.1 |
| Free drug (MMAE) | 0.050 | 97.7 |

TABLE 5B

| | ADC Cytotoxicity | |
|---|---|---|
| | on HCC-70 | |
| Abs | IC50 (nM) | Max Inh % |
| W3195-1.53.1-p1-uIgG1L-MMAE | 0.065 | 90.7 |
| WBP319-BMK4.uIgG1k-DM1 | 0.80 | 75.5 |
| Isotype control | >10 | 5.8 |
| Free payload control (MC-VC-PAB-MMAE) | >10 | 1.3 |
| Free drug (MMAE) | 0.23 | 87.2 |

TABLE 5C

| | ADC Cytotoxicity on HT-29 | |
|---|---|---|
| mAb | IC50 (nM) | Max Inh % |
| W3195-1.53.1-p1-uIgG1L-MMAE | >10 | −4.1 |
| WBP319-BMK4.uIgG1k-DM1 | >10 | −1.7 |
| Isotype control | >10 | 1.6 |
| Free payload control | >10 | −0.1 |
| (MC-VC-PAB-MMAE) | | |
| Free drug (MMAE) | 0.52 | 95.5 |

TABLE 5D

| | ADC toxicity on A549 | |
|---|---|---|
| Abs | IC50 (nM) | Max Inh % |
| W3195-1.53.1-p1-uIgG1L-MMAE | >10 | −7.8 |
| WBP319-BMK4.uIgG1k-DM1 | >10 | −7.3 |
| Isotype control | >10 | −0.4 |
| Free payload control (MC-VC-PAB-MMAE) | >10 | −5.4 |
| Free drug (MMAE) | 1.37 | 89.2 |

TABLE 5E

| | ADC toxicity on | |
|---|---|---|
| | MDA-MB-453 | |
| Abs | IC50 (nM) | Max Inh % |
| W3195-1.53.1-p1-uIgG1L-MMAE | >10 | 4.0 |
| WBP319-BMK4.uIgG1k-DM1 | >10 | −0.3 |
| Isotype control | >10 | 5.2 |
| Free payload control (MC-VC-PAB-MMAE) | >10 | −0.6 |
| Free drug (MMAE) | 1.28 | 96.7 |

TABLE 5F

| | ADC Cytotoxicity on | |
|---|---|---|
| | NCI-H1650 | |
| Abs | IC50 (nM) | Max Inh % |
| W3195-1.53.1-p1-uIgG1L-MMAE | 0.027 | 80 |
| WBP319-BMK4.uIgG1k-DM1 | 1.14 | 65 |
| Isotype control | >10 | 1 |
| Free payload control (MC-VC-PAB-MMAE) | >10 | 1 |
| Free drug (MMAE) | 0.15 | 81 |

### 3.4 Antibody Mediated Internalization Assay (High Content Screening, HCS)

Operetta CLS (PerkinElmer) is a high content imaging and analysis system that can collect and analyze images of samples with high speed and sensitivity. One day before the assay day, Poly-D-Lysine (PDL) was diluted to 8 μg/mL in DPBS (Hyclone, #SH30028.03) and added 100 μL/well into 96-well clear bottom black plates (Greinier, #655090). The PDL-coated plates were then incubated at 37° C. for 1 hour before discarding the supernatant. HCC-1954 (ATCC, CRL-2338) cells or NCI-H1650 (ATCC, CRL-5883) cells were plated at 18000 cells per well into the PDL-coated plates in 100 μL RPMI1640 complete medium (Gibco, #22400-089) containing 10% FBS (Hyclone, #SH30084.03). On Day 1, supernatants in the plates were discarded and antibodies serially diluted in 1% BSA/1×PBS (Ca+/Mg+) were added at 100 L/well and incubated at 4° C. for 2 hours. A non-related hlgG1 antibody was used as an isotype control.

For HCC-1954 cells, cells were washed with 100 μL 1% BSA/1×PBS (Ca+/Mg+) by Multi-channel Pipettes (Eppendorf) and then resuspended in PE conjugated Goat anti-human IgG Fc (Jackson, #109-115-098) 1:150 diluted in 1% BSA/1×PBS (Ca+/Mg+) for 1 hour at 4° C. in the dark. Cells were then washed one time as described above and resuspended in 1% BSA/1×PBS (Ca+/Mg+) for 2 hours at 37° C. Supernatants were discarded and 100 μL/well quench buffer (0.1 M glycine, 0.15 M NaCl, adjust pH to 2.5) were added and incubated at 4° C. for 5 min. Cells were then washed one time as described above and resuspended in Hoechst 33342 (Invitrogen, #H3570) 1:5000 diluted in DPBS (Hyclone, #SH30028.03) for 15 min at room temperature. After washed with DPBS (Hyclone, #SH30028.03) one time as described above, cells were resuspended in 4% PFA and stored at 4° C.

For NCI-H1650 cells, cells were washed with 100 μL 1% BSA/1×PBS (Ca+/Mg+) by Multi-channel Pipettes (Eppendorf) and then resuspended in Alexa647 conjugated Goat anti-human IgG Fc (Jackson, #109-605-098) 1:500 diluted in 1% BSA/1×PBS (Ca+/Mg+) for 1 hour at 4° C. in the dark. Cells were then washed one time as described above and resuspended in 1% BSA/1×PBS (Ca+/Mg+) for 2 hours at 37° C. Supernatants were discarded and cells were resuspended in Hoechst 33342 (Invitrogen, #H3570) 1:5000 diluted in DPBS (Hyclone, #SH30028.03) for 15 min at room temperature. After washed with DPBS (Hyclone, #SH30028.03) one time as described above, 100 μL/well quench buffer (0.1 M glycine, 0.15 M NaCl, adjust pH to 2.5) were added and incubated at 4° C. for 5 min. After washing for once with 1% BSA/1×PBS (Ca+/Mg+), cells were resuspended in 4% PFA and stored at 4° C.

Images were collected and analyzed by operetta CLS (PerkinElmer). The amount of internalized anti-P-cadherin antibody was assessed by measuring the mean fluorescence (MFI) per cell, wells containing no antibody or secondary antibody only were used to establish background fluorescence. Internalization EC50 values were obtained by the four-parameter non-linear regression analysis using GraphPad Prism 6 software.

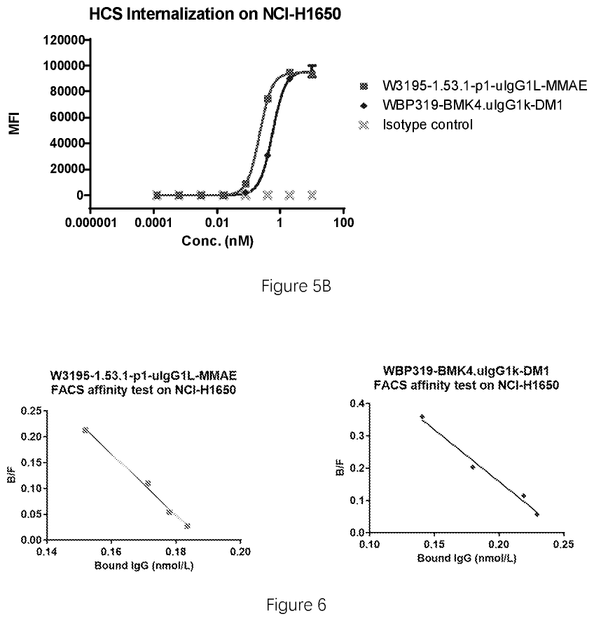

As shown in FIG. 5A and Table 6A, W3195-1.53.1-uIgG1L-MMAE showed good internalization ability with an EC50 of 0.023 nM using HCS assay, which was comparable with BMK4 (0.019 nM).

TABLE 6A
Internalization result of HCC-1954 cells in HCS assay

HCC-1954

| Abs | EC50 (nM) | Top MFI |
|---|---|---|
| W3195-1.53.1-p1-uIgG1L-MMAE | 0.023 | 1280 |
| W319-BMK4.uIgG1K | 0.019 | 1339 |
| Isotype control | >10 | 184 |

As shown in FIG. 5B and Table 6B, W3195-1.53.1-uIgG1L-MMAE showed good internalization ability with an EC50 of 0.22 nM using HCS assay, which was better than with the reference ADC WBP319-BMK4.uIgG1k-DM1 (0.57 nM).

TABLE 6B
Internalization result of NCI-H1650 cells in HCS assay

NCI-H1650

| Abs | EC50 (nM) | Top MFI |
|---|---|---|
| W3195-1.53.1-p1-uIgG1L-MMAE | 0.22 | 94928 |
| WBP319-BMK4.uIgG1k-DM1 | 0.57 | 89885 |
| Isotype control | N.A. | 18 |

### 3.5 Affinity to P-Cadherin (FACS)

The NCI-H1650 cells were seeded in 96-well U-bottom plates (BD) at a density of 5×10⁴ cells/well. Antibodies to be tested were serially diluted in 1×PBS/1% BSA and incubated with cells at 4° C. for 1 h. The plates were centrifuged and the supernatant was discarded. The cells were then incubated with Alexa647 conjugated goat anti-human IgG Fc (Jackson) at 4° C. in the dark for 30 min. After washing, the cells were re-suspended in 100 μL 1×PBS/1% BSA, and fluorescence intensity was measured by flow cytometry (BD Canto II) and analyzed by FlowJo software. The fluorescence intensity was converted to bound molecules/cell based on the quantitative beads standard curve (Quantum™ MESF Kits, Bangs Laboratories). KD was calculated by Graphpad Prism software.

The result is shown in the Table 7 and FIG. 6. W3195-1.53.1-p1-uIgG1L-MMAE showed good binding affinity on human P-cadherin expressing NCI-H1650 cells with a KD of 1.68E-10 M, which is comparable with W319-BMK4-uIgG1K-DM1 (3.11E-10 M).

TABLE 7
FACS affinity result of ADC to NCI-H1650 cells

| | W3195-1.53.1-p1- | |
|---|---|---|
| Sample | uIgG1L-MMAE | WBP319-BMK4.uIgG1k-DM1 |
| Bmax (M) | 1.88E−10 | 2.49E−10 |
| KD (M) | 1.68E−10 | 3.11E−10 |
| r² | 0.993 | 0.984 |

### 3.6 Domain Determination Binding (ELISA)

The binding domains of the antibody drug conjugates to human P-Cadherin extra cellular domain (ECD) were determined by a direct protein binding ELISA. 96-well high protein binding ELISA plates (Nunc) were coated overnight at 4° ° C. with antigens in coating buffer. All wells were washed three time with PBS/0.5%. Tween-20 (v/v) and all the following wash steps in the assay were performed the same. The wells were then blocked for one hour with 2% BSA (Bovogen)/1×PBS (Ca+/Mg+) (Gibco) and washed three times. For the primary antibody binding, the testing antibodies including the BMKs and our antibodies serially diluted in 2% BSA/1×PBS (Ca+/Mg+) were added to the relevant wells and incubated at ambient temperature for two hours. Plates were washed three times prior to the addition of 100 μL of secondary antibody Goat anti-Human-IgG-F(ab′)2-HRP (Jackson) diluted in 2% BSA/1×PBS (Ca+/Mg+). Plates were incubated at room temperature for one hour, followed by six washes as describe above.

For the binding detection, 100 μL Tetramethylbenzidine (TMB) Substrate solution (Invitrogen) was added to all wells before stopping the reaction with 100 μL 2M HCl. The extent of the testing Abs binding to P-Cadherin ECD (i.e. huCDH3 ECD, Uniprot ID: P22223 aa108-654), P-Cadherin ECD domain 1 (aa 108-236), P-Cadherin ECD domain 1+2 (aa 108-348), P-Cadherin ECD domain 1+2+3 (aa 108-461), P-Cadherin ECD domain 1+2+3+4 (aa 108-550) proteins were determined by measuring the OD450-OD540 absorbance using the SpectraMax® M5e microplate reader. EC50 values were obtained by the four-parameter non-linear regression analysis using GraphPad Prism software.

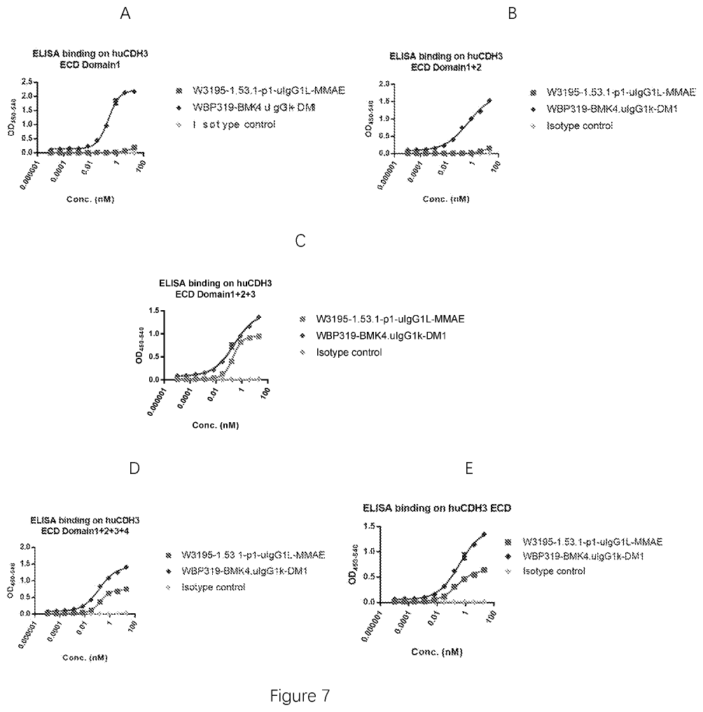

As indicated in Table 8 and FIGS. 7A-E, W3195-1.53.1-p1-uIgG1L-MMAE showed binding on Domain 3 (aa 348-461), which is different with WBP319-BMK4.uIgG1k-DM1 (Domain 1, aa 108-236).

TABLE 8
Domain determination binding result of ADCs

| | huCDH3 ECD | | huCDH3 ECD | | huCDH3 ECD | | huCDH3 ECD | | huCDH3 | |
|---|---|---|---|---|---|---|---|---|---|---|
| | Domain1 | | Domain1 + 2 | | Domain1 + 2 + 3 | | Domain1 + 2 + 3 + 4 | | ECD | |
| | EC50 | Max | EC50 | Max | EC50 | Max | EC50 | Max | EC50 | Max |
| Sample | (nM) | OD | (nM) | OD | (nM) | OD | (nM) | OD | (nM) | OD |
| W3195-1.53.1-p1- | N.A. | 0.19 | N.A. | 0.16 | 0.20 | 0.95 | 0.17 | 0.75 | 0.23 | 0.65 |
| uIgG1L-MMAE | | | | | | | | | | |
| WBP319-BMK4.uIgG1k- | 0.22 | 2.17 | 0.49 | 1.53 | 0.26 | 1.36 | 0.14 | 1.41 | 0.37 | 1.35 |
| DM1 | | | | | | | | | | |
| IgG1 isotype control | N.A. | 0.014 | N.A. | 0.014 | N.A. | 0.015 | N.A. | 0.03 | N.A. | 0.01 |

### 3.7 Non-Specific Binding (FACS)

Human cell lines were seeded in 96-well plates (BD) at a density of 1×10⁵ cells/well. 10 μg/mL antibody samples were added to cells and incubated for 1 h at 4° C. After washing, the cells were resuspended and incubated with PE-conjugated goat anti-human IgG Fc antibody (Jackson) for 30 min. After washing and resuspending, mean fluorescence intensity (MFI) was measured by flow cytometry (BD Canto II) and analyzed by FlowJo.

As shown in Table 9, W3195-1.53.1-p1-uIgG1L-MMAE showed no non-specific binding effect to all 12 human cell lines. WBP319-BMK4.uIgG1k-DM1 showed non-specific binding to HepG2 and 293F cell lines.

TABLE 9
Non-specific binding results by FACS

| | MFI | | | | | | |
|---|---|---|---|---|---|---|---|
| | W3195- | WBP319- | hIgG1K | hIgG1L | | | |
| | 1.53.1-p1- | BMK4.uIgG1k- | Isotype | Isotype | Positive | | |
| Cell/Sample | uIgG1L-MMAE | DM1 | control | control | Control | Blank | NC |
| Jurkat | 63.5 | 31.6 | 17.2 | 16.1 | 14700 | 15.7 | 18.8 |
| MDA-MB-453 | 47.4 | 63.5 | 19.6 | 19.5 | 1494 | 20.5 | 18.8 |
| HT29 | 26.4 | 22.7 | 20.5 | 20.2 | 2310 | 18.8 | 21.2 |
| HepG2 | 52.6 | 173 | 55 | 42.3 | 263 | 21.3 | 39.3 |
| SK-OV-3 | 30.6 | 27.4 | 24.3 | 23.8 | 3820 | 23.6 | 24.1 |
| Calu-6 | 38.5 | 61.2 | 18.8 | 18.6 | 3174 | 20.4 | 44.8 |
| A375 | 20.3 | 20.5 | 17.4 | 18.7 | 1818 | 19.7 | 18.1 |
| A204 | 29.4 | 69.8 | 27.4 | 26.7 | 5032 | 19.6 | 27.3 |
| 8505C | 26.5 | 41.2 | 21.7 | 20.8 | 2694 | 20.2 | 21.5 |
| H1 Hela | 27 | 43.5 | 24 | 23.3 | 426 | 21.1 | 22.4 |
| 293F | 35.1 | 90 | 17.1 | 17.9 | 2974 | 17.4 | 17.3 |
| BT474 | 34.6 | 45.6 | 19 | 18.3 | 3344 | 15.5 | 18.3 |

### Example 4

### In Vivo Antitumor Efficacy Study of ADCs

### 4.1 Xenografted HCC70 Breast Tumor Model-Study I

### Animal Preparation and Cell Culture for Implantation

WBP3195-p1-ADC in efficacy study was tested in HCC70 breast tumor model in SCID-17B female mice. Female SCID-17B mice (Beijing Vital River Lab Animal Technology Co. Ltd) of 28-29 weeks-old were used in the first study. HCC70 cells were maintained in vitro as a monolayer culture in RIPM1640 medium supplemented with 10% fetal bovine serum and 1% Penicillin-Streptomycin at 37° C. in an atmosphere of 5% CO2 in air. The tumor cells were routinely sub-cultured twice a week with 0.25% trypsin-EDTA treatment. The cells growing in an exponential growth phase were harvested and counted for tumor inoculation. The harvested cells were re-suspended in PBS at a density of 1.0×10⁸ cells/mL then added the equivalent matrigel (final 1.0×10⁷ cells/200 μL/mouse) with viability >90% and subcutaneously implanted into the right front flank of animals.

### Treatment and Data Collection

When the average tumor volume reached approximately to 230 mm³ at day 10 post inoculation, animals were randomly grouped into 3 groups and each group contained 7 mice. The 3 groups of mice received following intravenous injections at 1 mg/kg (single dose), respectively: Isotype control-MMAE, WBP319-BMK4.uIgG1k-DM1 and W3195-1.53.1-p1-uIgG1L-MMAE. The day of intravenous injection was considered as day 0. For all tumor studies, mice were weighed and tumor growth was measured twice a week using calipers.

### Study Guidance and Data Analysis

All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC). Tumor volume was calculated with the formula (½ (length×width²).

TGI (tumor growth inhibition) was calculated for each group using the formula: TGI (%)=[1−(Ti−T0)/(Vi−V0)]×100%. Ti is the average tumor volume of a treatment group on a given day. TO is the average tumor volume of the treatment group on the first day of treatment. Vi is the average tumor volume of the vehicle control group on the same day with Ti and V0 is the average tumor volume of the vehicle group on the first day of treatment. Relative change of bodyweight change (RCBW) was calculated with the formula [(BWt−BW0)/BW0]×100%, BW0 is the average bodyweight at day0, BWt is the average bodyweight at a measurement day. The results were represented by mean and the standard error (Mean±SEM). Data at day 34 were analyzed using Ordinary one-way ANOVA Tukey's multiple comparisons test with Graphpad Prism 6.0 and p<0.05 was considered as statistically significant.

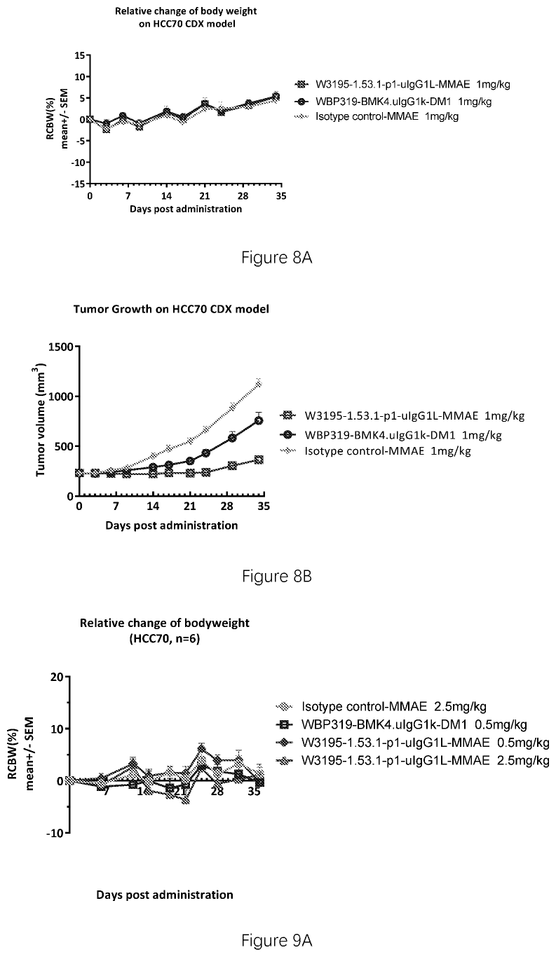

As shown in FIG. 8A, no obvious body weight loss was observed on all animals in each group, which indicated that animals were tolerated well to each test article.

At the 34 days post treatment, the mean tumor volume of isotype control group was 1120 mm³, which indicated HCC70 xenograft breast tumor model was well established. The TGI (%) at day 34 of each group was 40.91% for WBP319-BMK4.uIgG1k-DM1 and 85.15% for W3195-1.53.1-p1-uIgG1L-MMAE. Compared with isotype control group, all test articles significantly inhibited tumor growth; Comparing to WBP319-BMK4.uIgG1k-DM1, W3195-p1-MMAE showed more potent anti-tumor effect (p<0.01) (see FIG. 8B and Table 10).

TABLE 10
Comparison of HCC70 breast tumor inhibition effect

| Group | TGI (%), Day 34, significance |
|---|---|
| Isotype control-MMAE | vs Isotype control |
| WBP319-BMK4.uIgG1k-DM1 | 40.91**** |
| W3195-1.53.1-p1-uIgG1L-MMAE | 85.15**** |

Two-way RM ANOVA, * P < 0.05, ** P < 0.01, *** P < 0.001, ****P < 0.0001

### 4.2 Xenografted HCC70 Breast Tumor Model-Study II

### Animal Preparation and Cell Culture for Implantation

WBP3195-p1-ADC in efficacy study was tested in HCC70 breast tumor model in SCID-17B female mice. One hundred female SCID-17B mice (Beijing Vital River Lab Animal Technology Co. Ltd) of 37-38 weeks-old were used in the second study. HCC70 cells were maintained in vitro as a monolayer culture in RIPM1640 medium supplemented with 10% fetal bovine serum and 1% Penicillin-Sreptomycin at 37° C. in an atmosphere of 5% CO2 in air. The tumor cells were routinely sub-cultured twice a week with 0.25% trypsin-EDTA treatment. The cells growing in an exponential growth phase were harvested and counted for tumor inoculation. The harvested cells were re-suspended in PBS at a density of 1.0×10⁸ cells/mL then added the equivalent matrigel (final 1.0×10⁷ cells/200 μL/mouse) with viability >90% and subcutaneously implanted into the right front flank of animals.

### Treatment and Data Collection

When the average tumor volume reached approximately 242 mm³ at day 10 post inoculation, animals were randomly grouped into 4 groups and each group contained 7 mice. The 4 groups of mice received following intravenous injections (single dose), respectively: Isotype control-MMAE 2.5 mg/kg, WBP319-BMK4.uIgG1k-DM1 0.5 mg/kg, W3195-1.53.1-p1-uIgG1L-MMAE 0.5 mg/kg and 2.5 mg/kg. The day of intravenous injection was considered as day 0. For all tumor studies, mice were weighed and tumor growth was measured twice a week using calipers.

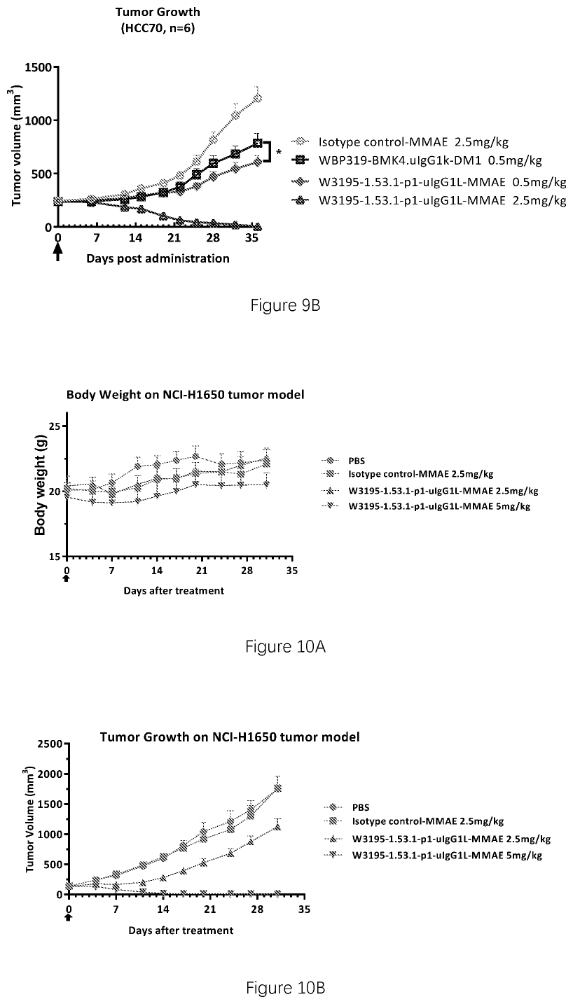

The data analysis procedures were conducted as described above. As shown in FIG. 9A, no obvious bodyweight loss was observed in all groups, which indicated that animals were tolerated well to each test articles.

At the 36 days post treatment, the mean tumor volume of isotype control group was 1202 mm³, which indicated HCC70 xenograft breast tumor model was well established. The TGI (%) at day 36 of each group was 42.69% for WBP319-BMK4.uIgG1k-DM1 0.5 mg/kg, 62.06% for W3195-1.53.1-p1-uIgG1L-MMAE 0.5 mg/kg and 122.67% for W3195-1.53.1-p1-uIgG1L-MMAE 2.5 mg/kg. Compared with isotype control group, all test articles significantly inhibited tumor growth; At 0.5 mg/kg, W3195-1.53.1-p1-uIgG1L-MMAE showed strong anti-tumor effect comparing to WBP319-BMK4.uIgG1k-DM1(p<0.05). At 2.5 mg/kg, 5 tumor free animals were observed in W3195-1.53.1-p1-uIgG1L-MMAE treatment group at day36 post treatment. The result indicated that W3195-p1-MMAE showed good dose-dependent antitumor effect at 0.5 mg/kg and 2.5 mg/kg (see FIG. 9B and Table 11).

TABLE 11
Summary of HCC70 breast tumor inhibition
effect at different doses

| | TGI (%), Day 36, | Tumor |
|---|---|---|
| Group | significance vs | free, |
| Isotype control-MMAE | Isotype control | n/total |
| WBP319-BMK4.uIgG1k-DM1, 0.5 mpk | 42.69**** | 0/6 |
| W3195-1.53.1-p1-uIgG1L-MMAE, 0.5 mpk | 62.06**** | 0/6 |
| W3195-1.53.1-p1-uIgG1L-MMAE, 2.5 mpk | 122.67**** | 5/6 |

Two-way RM ANOVA, * P < 0.05, ** P < 0.01, *** P < 0.001, ****P < 0.0001

### 4.3 Xenografted NCI-H1650 Lung Tumor Model

WBP3195-p1-ADC in efficacy study was tested in NCI-H1650 lung tumor model in SCID-17B female mice. Female SCID-17B mice (Shanghai Jihui Laboratory Animal Care Co., Ltd.) of 7-9 week-old were used in the study. NCI-H1650 cells were maintained in vitro as a monolayer culture in RIPM1640 medium supplemented with 10% fetal bovine serum, 100 U/mL penicillin and 100 μg/mL streptomycin at 37° C. in an atmosphere of 5% CO2 in air. The tumor cells were routinely sub-cultured twice a week with 0.25% trypsin-EDTA treatment. The cells growing in an exponential growth phase were harvested and counted for tumor inoculation.

For the therapeutic model, each mouse was inoculated subcutaneously at the right front flank with NCI-H1650 tumor cells (0.5×10⁷ cells resuspended in 100 ul PBS). When the average tumor volume reached approximately to 137 mm³ at day22 post inoculation, animals were randomly grouped into 4 groups and each group contained 6 mice. The 4 groups of mice received following intravenous injections with PBS, Isotype control-MMAE (2.5 mg/kg), W3195-1.53.1-p1-uIgG1L-MMAE (2.5 mg/kg) and W3195-1.53.1-p1-uIgG1L-MMAE (5 mg/kg) (single dose), respectively. The day of intravenous injection was considered as day 0. For all tumor studies, mice were weighed and tumor growth was measured twice a week using calipers.

All the procedures related to animal handling, care and the treatment in the study were performed according to the guidelines approved by the Institutional Animal Care and Use Committee (IACUC) of Shanghai SIPPR-BK Laboratory Animal Co., Ltd following the guidance of the Association for Assessment and Accreditation of Laboratory Animal Care (AAALAC). Tumor volume was calculated with the formula (½(length×width2). TGI (tumor growth inhibition) was calculated for each group using the formula: TGI (%)=[1-(Ti−T0)/(Vi−V0)]×100. Ti is the average tumor volume of a treatment group on a given day. TO is the average tumor volume of the treatment group at day0. Vi is the average tumor volume of the vehicle control group on the same day with Ti and V0 is the average tumor volume of the vehicle group at day0. Body weight was calculated with the formula. Data at day 31 were analyzed using two-way ANOVA Tukey's multiple comparisons test with Graphpad Prism 6.0 and p<0.05 was considered to be statistically significant.

No obvious bodyweight loss was observed on all the animals in each group, which indicated that animals were tolerated well to each test articles (FIG. 10A). At the 31 days post treatment, the mean tumor volume of PBS and Isotype control-MMAE groups were 1750.24±210.76 mm3 and 1762.31±197.71 mm3 respectively. The average tumor volume of mice treated with W3195-1.53.1-p1-uIgG1L-MMAE at 2.5 mg/kg was 1124.80+132.22 mm3 (TGI=38.69%), the average tumor volume of mice treated with W3195-1.53.1-p1-uIgG1L-MMAE at 5 mg/kg was 9.92+3.20 mm3 (TGI=107.99%). One tumor free animal was observed in W3195-1.53.1-p1-uIgG1L-MMAE (5 mg/kg) group (FIG. 10B and Table 12). Compared with PBS group, W3195-1.53.1-p1-uIgG1L-MMAE at high dose treatment showed obvious anti-tumor effect (P<0.0001), W3195-1.53.1-p1-uIgGIL-MMAE at low dose treatment showed partial anti-tumor effect (P<0.0001), and high dose is better than low dose group (p<0.0001). Dose dependent was observed between low dose and high dose group (p<0.0001), and one tumor free animal observed in high dose group.

TABLE 12
Summary of NCI-H1650 lung tumor inhibition effect

| | TGI (%) at Day 31 | Tumor free (TV <20 mm³) |
|---|---|---|
| Group (mg/kg) | significance with PBS | at Day 31, n/total |
| PBS | — | 0/6 |
| Isotype Control-MMAE 2.5 mg/kg | −0.75 | 0/6 |
| W3195-1.53.1-p1-uIgG1L-MMAE 2.5 mg/kg | 38.69**** | 0/6 |
| W3195-1.53.1-p1-uIgG1L-MMAE 5 mg/kg | 107.99**** | 5/6 |

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

1-26. (canceled)

27. An antibody-drug conjugate (ADC) comprising an antibody or antigen-binding portion thereof conjugated to a drug moiety, wherein the antibody or antigen-binding portion thereof binds P-cadherin and comprises:
a HCDR1 comprising the amino acid sequence of SEQ ID NO: 1;
a HCDR2 comprising the amino acid sequence of SEQ ID NO: 2;
a HCDR3 comprising the amino acid sequence of SEQ ID NO: 3;
a LCDR1 comprising the amino acid sequence of SEQ ID NO: 4;
a LCDR2 comprising the amino acid sequence of SEQ ID NO: 5; and
a LCDR3 comprising the amino acid sequence of SEQ ID NO: 6.

28. The ADC of claim 27, wherein the drug moiety comprises a cytotoxic agent or cytostatic agent selected from a toxin, a chemotherapeutic agent, an antibiotic, a radioactive isotope, or a nucleolytic enzyme.

29. The ADC of claim 28, wherein the cytotoxic agent is selected from maytansinoids (such as DM1, DM3, DM4), dolastatins, dolostatin peptidic analogs and derivatives thereof, such as auristatins, calicheamicin, trichothecene, and CC1065, optionally the cytotoxic agent is MMAE, DM1 or MMAF.

30. The ADC of claim 27, wherein the ADC has the formula Ab-(L-D)p, wherein Ab is the antibody or antigen-binding portion thereof, L is a linker, D is the drug moiety, and p is a integer from 1 to 20.

31. The ADC of claim 30, wherein the linker is cleavable by a protease.

32. The ADC of claim 30, wherein the linker is attached to the antibody through a thiol group on the antibody.

33. The ADC of claim 30, wherein the linker is selected from 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio) pentanoate (SPP), N-succinimidyl 4-(N-maleimidomethyl) cyclohexane-1 carboxylate (SMCC), N-Succinimidyl (4-iodo-acetyl) aminobenzoate (SIAB), and 6-maleimidocaproyl-valine-citrulline-p-aminobenyloxycarbonyl (MC-vc-PAB).

34. The ADC of claim 30, wherein the ADC has the formula Ab-(L-MMAE)p, and p ranges from 1 to 8.

35. The ADC of claim 34, wherein the linker is MC-vc-PAB.

36. The ADC of claim 27, wherein the antibody or antigen-binding portion thereof comprises:
(A) a heavy chain variable region (VH):
(i) comprising the amino acid sequence as set forth in SEQ ID NO: 7; or
(ii) comprising an amino acid sequence at least 85%, 90%, or 95% identical to the amino acid sequence as set forth in SEQ ID NO: 7 yet retaining the specific binding affinity to P-cadherin; and/or
(B) a light chain variable region (VL):
(i) comprising the amino acid sequence as set forth in SEQ ID NO: 8; or
(ii) comprising an amino acid sequence at least 85%, at least 90%, or at least 95% identical to the amino acid sequence as set forth in SEQ ID NO: 8 yet retaining the specific binding affinity to P-cadherin.

37. The ADC of claim 27, wherein the antibody or antigen-binding portion thereof further comprises a human IgG constant domain, including a human IgG1, IgG2, IgG3 or IgG4 constant domain.

38. The ADC of claim 37, wherein human IgG constant domain is a human IgG1 constant domain or a variant thereof.

39. The ADC of claim 27, wherein:
the heavy chain of the antibody comprises a heavy chain variable region as set forth in SEQ ID NO: 7, and a heavy chain constant region as set forth in SEQ ID NO: 9; and
the light chain of the antibody comprises a light chain variable region as set forth in SEQ ID NO: 8, and a light chain constant region as set forth in SEQ ID NO: 10.

40. A pharmaceutical composition comprising the ADC of claim 27 and a pharmaceutically acceptable carrier.

41. A method for producing the ADC of claim 27, comprising the steps of:
cultivating a host cell comprising a vector encoding the antibody or antigen-binding portion thereof under suitable conditions for the expression of the vector;
isolating the antibody or antigen-binding portion thereof from the host cell; and
reacting a nucleophilic group of a drug moiety with a linker reagent to form drug-linker intermediate D-L, and then reacting D-L with the antibody or antigen-binding portion thereof, alternatively, reacting the antibody with a linker reagent to form antibody-linker intermediate Ab-L, and then reacting Ab-L with an activated drug moiety D, whereby the antibody-drug conjugate is formed.

42. The method of claim 37, wherein the DAR of ADCs is in a range of about 1 to about 8, preferably is about 4.

43. A method for treating or preventing P-cadherin positive cancer in a subject, comprising administering an effective amount of the ADC of claim 27 to the subject.

44. The method of claim 43, wherein the cancer is selected from breast cancer, colorectal cancer, lung cancer, ovarian cancer, melanoma, bladder cancer, renal cell carcinoma, liver cancer, prostate cancer, stomach cancer, pancreatic cancer, cervical cancer, esophageal carcinoma, endometrial cancer, skin cancer, head and neck cancer, testis cancer, thyroid cancer, urothelial cancer, non-Hodgkin's lymphoma, chronic lymphocytic leukemia, diffuse large B-cell lymphoma, and multiple myeloma.

45. The method of claim 44, wherein the cancer is breast cancer, lung cancer or colorectal cancer.

46. A kit for treating or diagnosing cancer, comprising a container comprising the ADC of claim 27.
